(19) United States
(12) Patent Application Publication
(10) Pub. No.: US 2024/0366139 A1
(43) Pub. Date: Nov. 7, 2024

(54) SYSTEM AND METHOD FOR PROBABILISTIC SEARCH FOR R-PEAK DETECTION FROM ELECTROCARDIOGRAM

(71) Applicant: UNIVERSITY HEALTH NETWORK, Toronto (CA)

(72) Inventors: Yupeng TIAN, Toronto (CA); Milad LANKARANY, Toronto (CA)

(21) Appl. No.: 18/651,767

(22) Filed: May 1, 2024

Related U.S. Application Data
(60) Provisional application No. 63/463,533, filed on May 2, 2023.

(51) Int. Cl. A61B 5/352; A61B 5/00; A61B 5/024; A61B 5/308; A61B 5/332; A61B 5/364

(52) U.S. Cl. CPC A61B 5/352; A61B 5/02405; A61B 5/308; A61B 5/364; A61B 5/7203; A61B 5/332

(57) ABSTRACT

According to an aspect there is provided systems, methods, and non-transitory computer readable mediums with instructions for R-peak detection from electrocardiogram (ECG) stored thereon. The method includes pre-processing and smoothing a data stream, detecting R-peaks in an initial time interval, generating a predicted time point of a first new R-peak by predicting a probabilistic distribution of the new R-peak following a last R-peak in the initial time interval with History Dependent Inverse Gaussian (HDIG) distribution, detecting the first new R-peak by searching for the first new R-peak around the predicted time point of the first new R-peak in an adaptive searching interval, detecting remaining R-peaks iteratively, computing heart rate metrics using the detected R-peaks. Peaks with largest amplitudes passing the varying adaptive threshold are detected as the R-peaks. The heart rate metrics being one or more of average heart rate, accuracy, F1-score, sensitivity, precision, and heart rate variability.

### CROSS REFERENCE TO RELATED APPLICATION

The application claims all benefit of including priority to U.S. Provisional Patent Application No. 63/463,533, filed on May 2, 2023, and entitled “SYSTEM AND METHOD FOR PROBABILISTIC SEARCH FOR R-PEAK DETECTION FROM ELECTROCARDIOGRAM”, the entire contents of which is hereby incorporated by reference.

### TECHNICAL FIELD

This disclosure generally relates to the field of electrocardiogram signal analysis and more specifically to systems and methods using probabilistic search to accurately detect R-peaks in electrocardiograms.

### INTRODUCTION

The electrocardiogram (ECG) is a cardiovascular diagnostic procedure and a tool for clinical practice. Heart rate variability can be an important quantitative measure of cardiovascular regulation by the autonomic nervous system. Heart rate variability may be an accurate means of diagnosing, for example, intra-ventricular conduction disturbances and arrhythmias.

ECG R-peak detection can be useful for inferring health information. After R-peak detection, other features containing valuable medical information can be found within the signal adjacent or proximate to the detected R-peak (e.g., the P-wave, T-wave, PR interval, QRS complex, etc.). These features in ECG signals can be useful in health monitoring of ventricular disturbances, cardiac contractile dysfunction, arrhythmias and many other diseases (e.g., diabetes and Parkinson's disease). Of these significant features the R-peak may often be the most evident in the ECG. R-peak detection can thus be the first step for any further analysis, classification, and post-processing of the ECG signals. For patients with Parkinson's disease, for example, the standard deviation of the RR intervals can be significantly smaller than those in healthy subjects. ECG can also be used for patient monitoring during drug use, therapy, pre-surgery assessments, and public cardiac health surveillance (e.g., screening high-risk occupations and environmental factors).

Efficient recording and information inference methods of the ECG signal may be useful because of the ECG signal's importance to health monitoring and disease detection.

Textile sensors can be used for on-body bio-sensing because they can be light weight with long durability, and can be conveniently knitted into everyday objects (wrist rings, underwear, etc.). Wearable sensors (e.g., textile sensors) can be useful because they can record real-time ECG in a non-invasive and continuous manner. Daily health monitoring with wearable-recorded ECG signals can save time and medical costs and help detect diseases at an early stage. The early detection of potential health threats can lead to better treatment at lower cost with longer survival. Thus, wearable-recorded ECG signals can be beneficial in improving public health.

However, the quality of signals received from wearable-ECG can pose technical challenges (e.g., textile sensors) and, in particular, the quality of signals can make it difficult for detecting R-peaks. For example, accurate processing of ECG signals recorded using textile sensors can be challenging due to the very low signal-to-noise ratio (SNR) and a high level of motion artifacts. Motion artifacts are inevitable for signals recorded by textile sensors and may be the most challenging type of noise for ECG R-peak detection. Improvement in R-peak detection processes and systems, especially for textile-recorded ECG, is desirable.

### SUMMARY

In an aspect, embodiments described herein provide systems and methods for probabilistic search for R-peak detection from ECG. The R-peak detection can be used to compute heart rate metrics. The heart rate determination using the R-peak detection can be in real-time (e.g., discrete metrics), or may used for continuous heart rate monitoring.

The accurate detection of R-peaks may be useful in inferring health information from ECG signals. For example, accurate R-peak detection and heart rate measurement computation from wearable-recorded ECG can aid in heart health screening and detecting diseases in an early stage which may ultimately save lives and/or medical costs.

In clinical recordings, the subjects may be restricted in movements (e.g., they are required to lie down or sit still). In wearable recordings, there may be no movement restrictions (e.g., the users may move their body or even jog). The movements during ECG recordings can lead to motion artifacts in the signals, and the R-peak detection accuracy may be reduced. For R-peak detections, motion artifact can be a technically challenging problem.

The probabilistic characterization of the underlying R-peak distribution quantifies the underlying biophysical mechanism, and may be important in accurately tracking the instantaneous or discrete heart rate in an ECG signal. Thus, the consideration of the R-peak probabilistic distributions may shed light on detecting R-peaks in the optimal place to increase the R-peak detection accuracy. An RR interval is the distance between two adjacent R-peaks. The probabilistic consideration of RR intervals may provide more accurate and efficient R-peak detection.

In some embodiments, the Probabilistic Search (PS) can involve the following steps: I. Signal pre-processing and II. R-peak detection. The Probabilistic Search may enhance the accuracy and efficiency with which R-peaks can be found, especially in noisy ECG data with motion artifacts (e.g., from wearable textile sensors).

Step I. Signal pre-processing: The raw, noisy ECG data containing motion artifacts can first be band-pass filtered to reduce the baseline wander and high frequency noise, and the energy can be calculated to amplify the abrupt changes (including R-peaks and high-amplitude artifacts). The signal can then be convolved with a Gaussian kernel for smoothing. The number and amplitude of noise peaks can be reduced, and the signal-to-noise ratio (SNR) can further be increased.

Step II. R-peak detection: At each R-peak in the pre-processed signal, the distribution of the next R-peak can be estimated as the History Dependent Inverse Gaussian (HDIG) with real-time updated model parameters based on the recent R-peak history. A detailed local search can be conducted in a structured searching interval around the HDIG predicted time point for the next R-peak, which passes an adaptive threshold.

Embodiments described herein provide a system for probabilistic search for R-peak detection from ECG that involves a communication interface to receive a data stream comprising an ECG signal. The system has a hardware processor programmed with executable instructions for the R-peak detection and heart rate monitoring. In some embodiments, the hardware processor: pre-processes and smooths the data stream comprising the ECG signal to reduce noise in the data stream and amplify the ECG signal; detects R-peaks in an initial time interval of the pre-processed data stream; generates a predicted time point of a first new R-peak by predicting a probabilistic distribution of the new R-peak following a last R-peak in the initial time interval with History Dependent Inverse Gaussian (HDIG) distribution, wherein distribution parameters are updated in real-time based on recent R-peak history (e.g., the positions of the previously recent R-peaks); detects the first new R-peak in the data stream by searching for the first new R-peak around the predicted time point of the first new R-peak in an adaptive searching interval, wherein the peak with a largest amplitude passing a varying adaptive threshold is detected as the first new R-peak; and detects remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, wherein peaks with largest amplitudes passing the varying adaptive threshold are detected as the remaining R-peaks.

The detected R-peaks can be used (e.g., by a hardware processor) to compute heart rate metrics. For example, the heart rate metrics can be one or more of average heart rate, accuracy, F1-score, sensitivity and precision. Accordingly, embodiments described herein can use detected R-peaks to compute may include multiple measures. As noted, example measures include precision, sensitivity, F1-score (e.g., accuracy), and average heart rate. The first three measures can track the detection accuracy for each R-peak, while the average heart rate represents the average accuracy in a longer time interval (i.e., “beats per minute”). F1-score is the harmonic mean of precision and sensitivity, and can be used as a standardized accuracy measure.

According to an aspect there is provided a system for probabilistic search for R-peak detection from electrocardiogram (ECG). The system includes a communication interface to receive a data stream including an ECG signal, a hardware processor programmed with executable instructions for the R-peak detection and heart rate monitoring, non-transitory memory for storing the executable instructions for the R-peak detection, and an output device for transmitting and/or storing the heart rate metrics. The hardware processor pre-processes and smooths the data stream comprising the ECG signal to reduce noise in the data stream and amplify the ECG signal, detects R-peaks in an initial time interval of the pre-processed data stream, generates a predicted time point of a first new R-peak by predicting a probabilistic distribution of the new R-peak following a last R-peak in the initial time interval with History Dependent Inverse Gaussian (HDIG) distribution, detects the first new R-peak in the data stream by searching for the first new R-peak around the predicted time point of the first new R-peak in an adaptive searching interval, detects remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, compute heart rate metrics using the detected R-peaks. Distribution parameters are updated in real-time based on recent R-peak history. The peak with a largest amplitude passing a varying adaptive threshold is detected as the first new R-peak. Peaks with largest amplitudes passing the varying adaptive threshold are detected as the remaining R-peaks. The heart rate metrics are one or more of average heart rate, accuracy, F1-score, sensitivity, precision, and heart rate variability.

According to a further aspect, the hardware processor measures the average heart rate in the data stream as a total number of detected R-peaks in the data stream divided by a time length of the data stream.

According to a further aspect, the hardware processor is programmed with executable instructions for continuous heart rate monitoring by continuously receiving the data stream and re-computing the heart rate metrics.

According to a further aspect, the system further includes a band-pass filter to pre-process the data stream to reduce baseline wander and high frequency noise.

According to a further aspect, the hardware processor pre-processes and smooths the data stream using an energy calculator that amplifies the R-peaks in the ECG signal.

According to a further aspect, the hardware processor pre-processes and smooths the data stream using a smoothing function that convolves the data stream with a Gaussian kernel specific to a sampling frequency of the data stream and a type of sensor recording the data stream to further increase the signal-to-noise ratio (SNR).

According to a further aspect, the hardware processor pre-processes and smooths the data stream with a Gaussian kernel by assigning higher weights to R-peaks in the ECG signal and lower weights to noise in the data stream.

According to a further aspect, distribution parameters are updated in real-time based on recent R-peak history with a fast optimization technique such that more recent R-peak history has higher weights than more previous R-peak history.

According to a further aspect, the adaptive searching interval depends on the predicted time point of the first new R-peak.

According to a further aspect, the adaptive searching interval depends on patient specific skew parameters based on an extent of arrhythmia selected from the group consisting of slight arrhythmia, moderate arrhythmia, and serious arrhythmia.

According to a further aspect, the adaptive searching interval is at an interval of increasing length.

According to a further aspect, the varying adaptive threshold is specific to a type of sensor recording the data stream.

According to a further aspect, the adaptive searching interval is generated by training on datasets based on a large amount of ECG data.

According to a further aspect, the system includes one or more electrodes to perform measurements to record the raw ECG signal.

According to a further aspect, the electrodes are dry-electrodes.

According to a further aspect, the electrodes are gel-electrodes.

According to a further aspect, the system includes one or more wearable sensors to perform measurements to record the raw ECG signal.

According to a further aspect, the one or more wearable sensors comprise one or more wearable textile sensors.

According to a further aspect, the HDIG distribution extracts an underlying probabilistic structure of R-peak positions in the data stream based on deterministic morphological features of ECG signals.

According to a further aspect, model parameters for the HDIG distribution are updated in real-time.

According to an aspect there is provided a method for probabilistic search for R-peak detection from electrocardiogram (ECG). The method includes pre-processing and smoothing a data stream comprising the ECG signal to reduce noise in the data stream and amplify the ECG signal, detecting R-peaks in an initial time interval of the pre-processed data stream, generating a predicted time point of a first new R-peak by predicting a probabilistic distribution of the new R-peak following a last R-peak in the initial time interval with History Dependent Inverse Gaussian (HDIG) distribution, detecting the first new R-peak in the data stream by searching for the first new R-peak around the predicted time point of the first new R-peak in an adaptive searching interval, detecting remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, computing heart rate metrics using the detected R-peaks. Distribution parameters are updated in real-time based on recent R-peak history. The peak with a largest amplitude passing a varying adaptive threshold is detected as the first new R-peak. Peaks with largest amplitudes passing the varying adaptive threshold are detected as the remaining R-peaks. The heart rate metrics being one or more of average heart rate, accuracy, F1-score, sensitivity, precision, and heart rate variability.

According to a further aspect, the method includes measuring the average heart rate in the data stream as a total number of detected R-peaks in the data stream divided by a time length of the data stream.

According to a further aspect, the method includes continuously processing the data stream and re-computing the heart rate metrics.

According to a further aspect, the preprocessing and smoothing includes band-pass filtering the data stream to reduce baseline wander and high frequency noise.

According to a further aspect, the preprocessing and smoothing uses an energy calculator that amplifies the R-peaks in the ECG signal.

According to a further aspect, the preprocessing and smoothing uses a smoothing function that convolves the data stream with a Gaussian kernel specific to a sampling frequency of the data stream and a type of sensor recording the data stream to further increase the signal-to-noise ratio (SNR).

According to a further aspect, the preprocessing and smoothing uses a Gaussian kernel by assigning higher weights to R-peaks in the ECG signal and lower weights to noise in the data stream.

According to a further aspect, distribution parameters are updated in real-time based on recent R-peak history with a fast optimization technique such that more recent R-peak history has higher weights than more previous R-peak history.

According to a further aspect, the adaptive searching interval depends on the predicted time point of the first new R-peak.

According to a further aspect, the adaptive searching interval depends on patient specific skew parameters based on an extent of arrhythmia selected from the group consisting of slight arrhythmia, moderate arrhythmia, and serious arrhythmia.

According to a further aspect, the adaptive searching interval is at an interval of increasing length.

According to a further aspect, the varying adaptive threshold is specific to a type of sensor recording the data stream.

According to a further aspect, the adaptive searching interval is generated by training on datasets based on a large amount of ECG data.

According to a further aspect, the data stream includes input from one or more electrodes performing measurements to record the raw ECG signal.

According to a further aspect, the electrodes are dry-electrodes.

According to a further aspect, the electrodes are gel-electrodes.

According to a further aspect, the data stream includes input from one or more wearable sensors performing measurements to record the raw ECG signal.

According to a further aspect, the one or more wearable sensors include one or more wearable textile sensors.

According to a further aspect, the HDIG distribution extracts an underlying probabilistic structure of R-peak positions in the data stream based on deterministic morphological features of ECG signals.

According to a further aspect, model parameters for the HDIG distribution are updated in real-time.

According to an aspect there is provided a non-transitory computer readable medium with instructions for R-peak detection from electrocardiogram (ECG) stored thereon. When executed by a hardware processor cause the processor to pre-process and smooth a data stream comprising the ECG signal to reduce noise in the data stream and amplify the ECG signal detect R-peaks in an initial time interval of the pre-processed data stream, generate a predicted time point of a first new R-peak by predicting a probabilistic distribution of the new R-peak following a last R-peak in the initial time interval with History Dependent Inverse Gaussian (HDIG) distribution, detect the first new R-peak in the data stream by searching for the first new R-peak around the predicted time point of the first new R-peak in an adaptive searching interval, detect remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, compute heart rate metrics using the detected R-peaks, the heart rate metrics being one or more of average heart rate, accuracy, F1-score, sensitivity, precision, and heart rate variability. Distribution parameters are updated in real-time based on recent R-peak history. The peak with a largest amplitude passing a varying adaptive threshold is detected as the first new R-peak. Peaks with largest amplitudes passing the varying adaptive threshold are detected as the remaining R-peaks.

According to a further aspect, the instructions cause the processor to measure the average heart rate in the data stream as a total number of detected R-peaks in the data stream divided by a time length of the data stream.

According to a further aspect, the instructions cause the processor to continuously process the data stream and re-compute the heart rate metrics.

According to a further aspect, the preprocessing and smoothing comprises band-pass filtering the data stream to reduce baseline wander and high frequency noise.

According to a further aspect, the preprocessing and smoothing uses an energy calculator that amplifies the R-peaks in the ECG signal.

According to a further aspect, the preprocessing and smoothing uses a smoothing function that convolves the data stream with a Gaussian kernel specific to a sampling frequency of the data stream and a type of sensor recording the data stream to further increase the signal-to-noise ratio (SNR).

According to a further aspect, the preprocessing and smoothing uses a Gaussian kernel by assigning higher weights to R-peaks in the ECG signal and lower weights to noise in the data stream.

According to a further aspect, distribution parameters are updated in real-time based on recent R-peak history with a fast optimization technique such that more recent R-peak history has higher weights than more previous R-peak history.

According to a further aspect, the adaptive searching interval depends on the predicted time point of the first new R-peak.

According to a further aspect, the adaptive searching interval depends on patient specific skew parameters based on an extent of arrhythmia selected from the group consisting of slight arrhythmia, moderate arrhythmia, and serious arrhythmia.

According to a further aspect, the adaptive searching interval is at an interval of increasing length.

According to a further aspect, the varying adaptive threshold is specific to a type of sensor recording the data stream.

According to a further aspect, the adaptive searching interval is generated by training on datasets based on a large amount of ECG data.

According to a further aspect, the data stream includes input from one or more electrodes performing measurements to record the raw ECG signal.

According to a further aspect, the electrodes are dry-electrodes.

According to a further aspect, the electrodes are gel-electrodes.

According to a further aspect, the data stream includes input from one or more wearable sensors performing measurements to record the raw ECG signal.

According to a further aspect, the one or more wearable sensors comprise one or more wearable textile sensors.

According to a further aspect, the HDIG distribution extracts an underlying probabilistic structure of R-peak positions in the data stream based on deterministic morphological features of ECG signals.

According to a further aspect, model parameters for the HDIG distribution are updated in real-time.

### DESCRIPTION OF THE FIGURES

In the figures,

FIG. 1A and FIG. 1B illustrate sample ECG signal segments.

FIG. 2 illustrates three example samples of a textile waist-recorded ECG in 3 statuses: sitting, standing, and jogging.

FIG. 3 illustrates a schematic summary of the Probabilistic Search (PS) method described herein, according to some embodiments.

FIG. 4 illustrates another example method of signal pre-processing and smoothing, according to some embodiments.

FIG. 5A illustrates a sample distribution of RR intervals in 30 minutes, according to some embodiments.

FIG. 5B illustrates an Inverse Gaussian distribution, according to some embodiments.

FIG. 6 illustrates an example system for detecting a next peak using probabilistic distribution, according to some embodiments.

FIG. 7 illustrates an example method for detecting a next peak using probabilistic distribution, according to some embodiments.

FIG. 8 illustrates a comparison in 13 s segments of the recording “MITDB recording 108 and Middle SNR textile noise” in the generated MITDB dataset between the local average method and smoothing method described herein, according to some embodiments.

FIG. 9 illustrates a Probabilistic Search (PS) method with a simplified searching interval, according to some embodiments.

FIG. 10 illustrates a construction of the searching intervals used in the Probabilistic Search (PS) method, according to some embodiments.

FIG. 11 illustrates an example method that summarizes the R-peak detection step of the Probabilistic Search (PS) method, according to some embodiments.

FIG. 12 illustrates the textile-like MITDB recordings, according to some embodiments.

FIG. 13 illustrates comparisons of precision, sensitivity, and F1-score of the other ECG R-peak detection methods with the example Probabilistic Search (PS) method in standardized Textile-like MITDB.

FIG. 14 illustrates a comparison of the precision (%) values of R-peak detection methods, according to some embodiments.

FIG. 15 illustrates a comparison of the sensitivity (%) values of R-peak detection methods, according to some embodiments.

FIG. 16 illustrates F1-score (%) values of R-peak detection methods, according to some embodiments.

FIG. 17 illustrates comparisons of heart rate estimate absolute error of the other ECG R-peak detection methods with the example Probabilistic Search (PS) method in the standardized Textile-like MITDB, according to some embodiments.

FIG. 18 illustrates the average heart rate estimate absolute error (bpm) in the whole Textile-like MITDB, according to some embodiments.

FIG. 19 illustrates the textile-ECG dataset and the reference chest R-peaks, according to some embodiments.

FIG. 20 illustrates the comparisons of both mean and standard deviation of the F1-score, according to some embodiments.

FIG. 21 is a schematic diagram of a computing device of FIG. 6 that can be used to implement aspects embodiments described herein.

### DETAILED DESCRIPTION

Embodiments described herein relate to systems and methods for probabilistic search for R-peak detection from electrocardiogram (ECG). The accurate detection of R-peaks may be useful in inferring health information from ECG signals, for example.

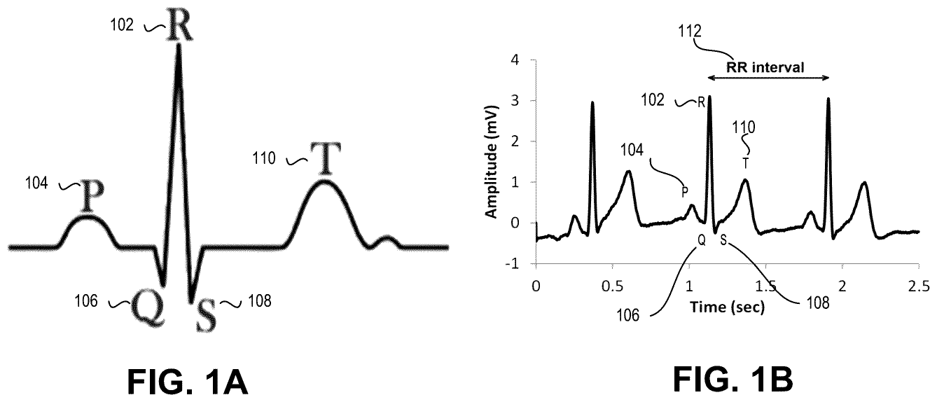

FIG. 1A and FIG. 1 B illustrate sample ECG signal segments. After R-peak 102 detection, other features containing valuable medical information can be found adjacent to it such as the P-wave 104, T-wave 110, QRS complex 106, 102, 108, respectively, PR interval, and so on. These significant features can be useful for health monitoring of many diseases. R-peak detection can be used to compute heart rate metrics, such as average heart rate, accuracy, F1-score, sensitivity and precision. Embodiments described herein can use detected R-peaks to compute may include multiple measurements. The precision, sensitivity, and F1-score measurements can track the detection accuracy for each R-peak, while the average heart rate can represent the average accuracy in a longer time interval (i.e., “beats per minute”). F1-score is the harmonic mean of precision and sensitivity, and can be used as a standardized accuracy measure.

As another example, R-peak detection can be used to compute heart rate variability. Further, R-peak detection can be used for further analysis and post-processing of the ECG signals and heart-related diseases.

Wearable sensors (e.g., textile sensors) can record ECG signals in a non-invasive and continuous manner on a day-to-day basis. However, textile-ECG signal analysis may be challenging because of the low signal-to-noise ratio and a high number of motion artifacts, especially in walking and jogging recordings. Embodiments described herein involve a Probabilistic Search (PS) method wherein the R-peak detection accuracy can significantly outperform other methods in textile-based ECG datasets using the RR interval 112.

There may be different sources of ECG signals, such as, for example: Clinically recorded ECG data (e.g., recorded in hospital with expensive devices and gel-electrodes); and ECG data recorded with wearable sensors (e.g., textile or metal sensors) and dry electrodes.

Clinically recorded ECGs can be clean with clear R-peaks, which can be detected with the Pan & Thompkins (PT) method. ECG recorded with wearable sensors can arise from sources such as wearable sensor data and textile-recorded ECG signals. Wearable sensor ECGs may have much lower signal-to-noise ratio (SNR) than clinical ECGs. R-peak detection can be challenging for such noisy ECG signals.

Accurate R-peak detection and heart metric computation (e.g., heart rate) from wearable-recorded ECG can aid in heart health screening and detecting diseases in an early stage which may ultimately save lives and/or medical costs.

In clinical recordings, the subjects may be restricted in movements (e.g., they may be required to lie down or sit still). In wearable recordings, there may be no movement restrictions (e.g., the users may move their body or even jog). Moving during ECG recordings can lead to motion artifacts in the signals and the R-peak detection accuracy may be reduced. For R-peak detections, motion artifacts can be technically challenging. However, heart function can sometimes be more clearly elucidated during movements (e.g., jogging), thus it would be beneficial to enable the use of wearable sensor while moving in spite of this low SNR and these motion artifacts.

Other R-peak detection processes may not be efficient for detecting R-peaks from wearable ECG. The accuracy of these other methods may drop from, for example, ˜99% (for clean ECG signals) to, for example, <80% (for textile-recorded ECG with motion artifacts).

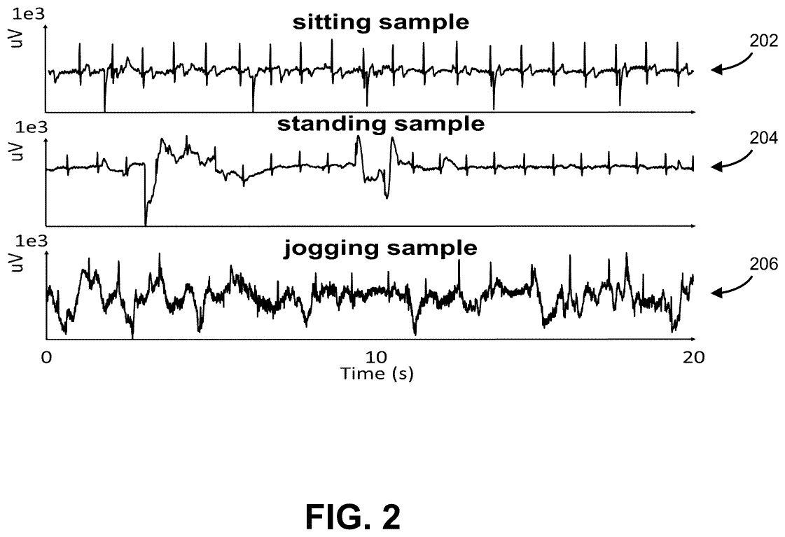

FIG. 2 illustrates three example samples of a textile waist-recorded ECG in 3 statuses: sitting (202), standing (204), and jogging (206), according to some embodiments. As can be seen in FIG. 2, the jogging sample (206) has a low SNR and is contaminated with motion artifacts as compared to the other statuses.

PT and other R-peak detection methods may work for clinically recorded. ECG signals can be recorded using gel-electrodes to generate datasets (e.g., the MIT-BIH Arrhythmia Database (MITDB)). MITDB is a standardized dataset to validate the performance of R-peak detection methods on high quality clinical ECG signals recorded by gel-electrode. Clinical ECG may have a much higher SNR (and fewer motion artifacts) than wearable-ECG and may mostly be recorded with elaborate and costly devices (which may not be brought home from, for example, the hospital or clinic). The standard clinical ECG includes recordings from 12 leads: 3 leads in an arm, 3 leads in a leg, and 6 precordial leads. Physicians use Holter monitor studies with 24-72 h of continuous ECG recordings, which may be laborious for patients, especially the elderly. Frequent hospital visits can be costly and time consuming. Portable out-of-hospital ECG recording devices can be used personally, but for such devices, their diagnostic accuracy, reproducibility, or utility may be limited. Thus, clinical ECG may be less accessible in daily life. There exists a need for an efficient R-peak detection process that can be compatible with wearable-ECG, such as textile-ECG, to help with day-to-day health monitoring and early disease detection.

However, PT and the other R-peak detection processes may not be efficient in ECG signals with prevalent textile noise, especially motion artifacts. PT and other methods consider the deterministic morphological features of ECG signals (e.g., peak amplitudes and signal frequency components). Such a deterministic morphological perspective may ignore the physiological mechanisms that generate R-peaks. This mechanism may be essentially probabilistic. The probabilistic characterization of the underlying R-peak distribution quantifies the underlying biophysical mechanism, and may be important in accurately tracking the instantaneous heart rate in an ECG signal. Thus, the consideration of the R-peak probabilistic distributions may shed light on detecting R-peaks in the optimal place to increase the R-peak detection accuracy.

An RR interval is the distance between two adjacent R-peaks, and is the electrical signal that can represent the ventricular contraction in the heart's conduction system. The R-peak can be generated by the synchronous depolarization of the heart's pacemaker cells beginning from the sinoatrial (SA) node of the right atrium, and then propagating to the left atrium and the two ventricles. The membrane potential of the heart's pacemaker cell can be depolarized by ion currents (e.g., Ca²⁺, K⁺, and Na⁺) to pass a threshold and trigger an action potential that can then propagate through the heart's conduction system. Such processes of the membrane potential dynamics can be modelled as a random walk stochastic process with a firing threshold and a drifting term to an equilibrium. The first passage time (FPT) means the time difference between two adjacent threshold-crossing events. The FPT probability density of such an integrate-and-fire drifting random walk process is derived as an Inverse Gaussian distribution. For an ECG signal, FPT represents the RR interval. Thus, Inverse Gaussian distributions can be used to study heart beats. The length of an RR interval can depend on the inputs from the autonomic nervous system (sympathetic and parasympathetic) to the SA node, and can be affected by the dynamics within the cardiovascular circuits. The effect of these inputs to the SA nodes can last for several seconds and thus, the RR interval lengths may not be independent but can depend on the recent R-peak history. Based on the above physiological considerations, the History Dependent Inverse Gaussian (HDIG) distribution can be used to realistically model RR intervals. The model states that RR intervals can follow the HDIG distribution, and the parameters in the distribution can depend on the recent R-peak history.

With the probabilistic characterization of RR interval distribution as HDIG, the Probabilistic Search (PS) R-peak detection method can be developed to efficiently find the R-peaks in ECG signals contaminated with noise (e.g., motion artifacts). The PS method can consist of two main steps: (i) signal pre-processing; and (ii) R-peak detection. In the signal pre-processing step, the raw ECG signal can first be passed through band-pass filters and an energy calculator. Then, to further reduce the SNR, the resulting signal can be smoothed by developing a convolution technique with a Gaussian kernel. Other methods may use a linear kernel (moving average method) for smoothing, and such methods may be blind in discriminating signal and noise. The non-linear method described herein may be more efficient in amplifying the SNR by stressing the R-peaks and suppressing the noise. After signal pre-processing, the smoothed signal can be used for R-peak detection. HDIG is used to modify the current (new observation) RR interval based on the previous (history) RR intervals. Subsequent to existing R-peaks, the new R-peak can be searched for around the mean of its HDIG distribution, which can be considered as the optimal time point to most probably find the R-peak. Around this optimal time point, a detailed adaptive local search methodology can be developed to accurately locate the R-peak. An evolving searching interval with gradual increments can be developed to reduce false positives and an appropriate skew parameter to reduce false negatives. The searching interval can be designed to increase the R-peak detection efficacy for both healthy subjects and arrhythmia patients. The highest peak in the searching interval passing an adaptive threshold can be chosen as the next R-peak. The PT adaptive threshold method can be improved by fitting to the searching interval structure and discovering specific parameters for textile-ECG.

The PS method described herein can be designed for automatic and accurate ECG R-peak detection and heart rate monitoring. The source of the ECG signal can be unrestricted. The method can be robust and accurate in both clinically recorded ECG (with gel-electrode) and ECG recorded by wearable sensors (with dry-electrode, e.g., textile sensors). ECG recorded by dry-electrodes may have a much lower signal-to-noise ratio (SNR) and higher level of motion artifacts. The proposed Probabilistic Search (PS) method can: (1) efficiently smooth ECG signals with the Gaussian kernel (e.g., increase the SNR), (2) predict the most probable time point that the next R-peak will happen, and (3) search for R-peaks and avoid artifacts in searching intervals with the multiple search design, adaptive thresholds, and skew parameters. The underlying probabilistic structure of R-peak positions can be extracted: a point process characterized by the HDIG model, and the model parameters can be updated in real-time. This method makes use of probabilistic considerations beyond the deterministic morphological features considered in other approaches. A detailed search for the next R-peaks can be conducted in intervals with increasing length and varying adaptive thresholds. The methods described herein can use patient-specific considerations to enhance the possible accuracy and efficiency. For example, the skew parameter τ (τ≥1) (described in greater detail below) of the adaptive searching interval can be bigger for people with high heart rate variability (e.g., arrhythmia) to compensate for some missed R-peaks (false negatives). As a further example, in practice, if a person tracks his/her clinical-ECG and finds that the heart rate variability (e.g., variance) is high, τ may be tuned a little higher (τ≤1.5 can be recommended). However, the algorithm may be robust to τ: for each person, the accuracy of the algorithm can be consistent for 1≤τ≤1.5; the tuning of T can slightly improve the accuracy. With the PS method, R-peak detection accuracy can be improved in ECG recorded by wearable sensors using dry-electrodes, and can achieve ˜100% using gel-electrodes, in some embodiments. In textile-recorded ECG with serious motion artifacts, the method can be >10% higher in R-peak detection F1-score than the other methods. In particular, the heart rate estimate can be very accurate. In >50 hours of ECG data with prevalent textile noise (e.g., a low SNR and many motion artifacts), the average absolute difference from true heart rate may be <1.3 bpm. Accurate R-peak detection and heart rate monitoring from wearable-recorded ECG can be important for heart health screening and detecting diseases in an early stage, which may save lives and medical costs.

The PS method can operate by first pre-processing and smoothing the raw ECG signal, and then doing a detailed local search for the next R-peak around the most probable place that the next R-peak will happen.

At each R-peak, the distribution of the next R-peak position can be modelled as a HDIG, where the distribution parameters can be accurately predicted based on the recent R-peak history. Such a history dependent real-time parameter update may be consistent with physiological reality because the effects of the parasympathetic and sympathetic inputs to the SA nodes (the pacemaker cells in heart) persist for several seconds. At each R-peak, the next R-peak can be searched for around the position predicted by HDIG. In the searching interval design, a multiple search structure and patient-specific skew parameters can be developed. The search can be done to accurately locate the R-peaks in an interval of increasing length with varying adaptive thresholds. For arrhythmia patients, the search may use a left-skewed searching interval (left-half>right-half) and the skew parameter can depend on the extent of arrhythmia. In practice, using 3 levels of skew can improve the R-peak prediction accuracy. The levels can be defined as (1) healthy/slight arrhythmia, (2) moderate arrhythmia, and (3) serious arrhythmia. The decisions of the searching interval structure can be trained on datasets based on large amounts of ECG data recorded by textile-sensors.

### Exemplary Probabilistic Search ECG R-Peak Detection Method

An exemplary R-peak detection method can be robust to the high levels of noise (in particular to motion artifacts) prevalent in ECG signals recorded by wearable sensors using probabilistic search. The method can be developed based on textile-ECG (i.e., with motion artifacts, a prevalent feature of the dry textile electrodes). PT and other methods may be less effective in R-peak detection for textile-ECG. The main reason of such possible failures lies in the lack of discrimination of the more probable places to search for R-peaks. As a result, these methods may select most of the peaks passing the triggering thresholds as R-peaks, possibly increasing the number of false positives (FP). In particular, when implementing these methods on wearable-ECG, the amount of FP may be large because of the prevalence of large noise whose amplitude may be comparable or much larger than the R-peaks. Thus, it can be important to develop an ECG R-peak detection method that incorporates the underlying R-peak probability distribution, and an effective strategy to accurately search for the R-peaks while avoiding the noise. The PS method can be developed based on such a probabilistic search strategy that accurately locates the R-peaks around the more probable place with an optimal searching method.

In the PS method, the raw ECG signal is first pre-processed before the R-peak detections. The signal pre-processing step can consist of 3 sub-steps: (1) median filter and bandpass filter; (2) derivative and energy calculations; (3) smoothing with convolution. A smoothing technique can be developed with a Gaussian kernel that more effectively increases the SNR than the current approaches. After the signal pre-processing, the SNR can be increased. R-peaks can be detected in the pre-processed ECG, and the R-peak detection consists of 2 sub-steps: (1) prediction; and (2) search. To detect a new R-peak, its probabilistic distribution first estimate can be based on the preceding R-peaks with HDIG. The HDIG probabilistic density function can be defined by the following:

$$f ( t \vert {H}_{{u}_{k}} , θ ) = {[ \frac{{θ}_{p + 1}}{2 {π ( t - {u}_{k} )}^{3}} ]}^{\frac{1}{2}} * exp \{ - \frac{1}{2} * \frac{{{θ}_{p + 1} [ t - {u}_{k} - μ ( {H}_{{u}_{k}} , θ ) ]}^{2}}{{μ ( {H}_{{u}_{k}} , θ )}^{2} ( t - {u}_{k} )} \} \qquad ( 1 )$$

where Hᵤ_{ₖ} can be the vector containing the time point of the current R-peak (uₖ) and its preceding history RR intervals, θ can be the time-varying parameter vector, μ(Hᵤ_{ₖ}, B) can be the mean of the HDIG distribution, and θₚ₊₁>0 can be the shape parameter of the HDIG distribution. The HDIG model parameters θ can be optimized with a real-time trust region method. An optimal search method can be developed around the HDIG predicted time point (uₖ+μ(Hᵤ_{ₖ}, θ)) for the next R-peak. A local searching interval with an evolving length can be used to decrease the number of false positive detections. A skew parameter can be added to the searching interval to decrease the number of false negatives (i.e., missed R-peaks). The skew parameter may be different for healthy subjects and arrhythmia patients to maximize the R-peak detection accuracy. Within the searching interval, the maximal peak passing an adaptive threshold can be chosen as the new R-peak.

In some training embodiments, the detected R-peaks can optionally be compared with an annotation file that records the true R-peak time points. This can be used to train and improve the system.

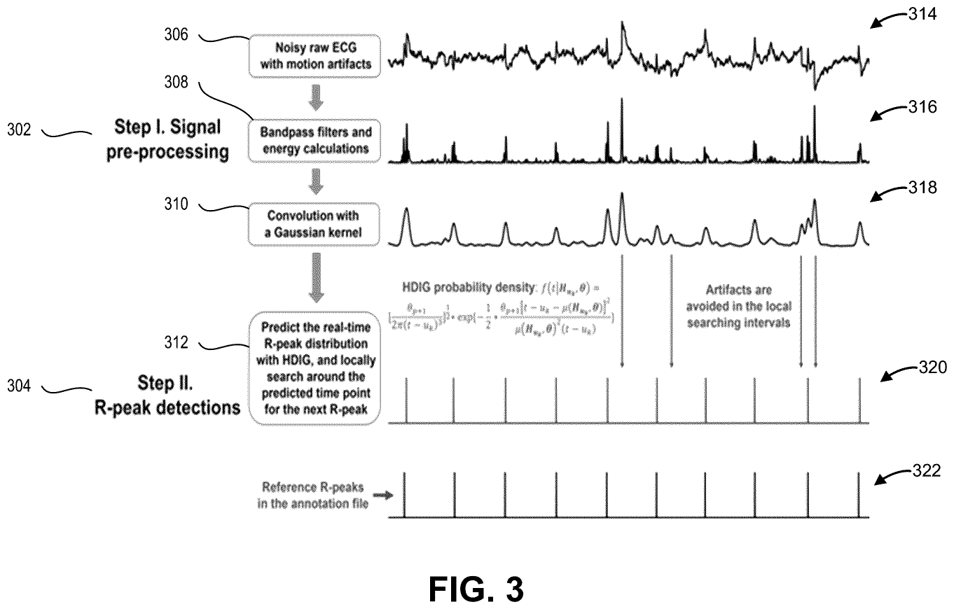

FIG. 3 illustrates a schematic summary of the Probabilistic Search (PS) method described herein, according to some embodiments.

The PS method can consist of two steps: I. Signal pre-processing (302) and II. R-peak detection (304).

Step I. Signal pre-processing (302): The raw, noisy ECG data (306; 314) containing motion artifacts can first be band-pass filtered to reduce the baseline wanders and high frequency noise, and the energy can be calculated to amplify the abrupt changes (including R-peaks and high-amplitude artifacts) (308) to generate 316. The signal can then be convolved with a Gaussian kernel for smoothing (310) to generate 318. The number and amplitude of noise peaks are reduced and the signal-to-noise ratio (SNR) can further be increased.

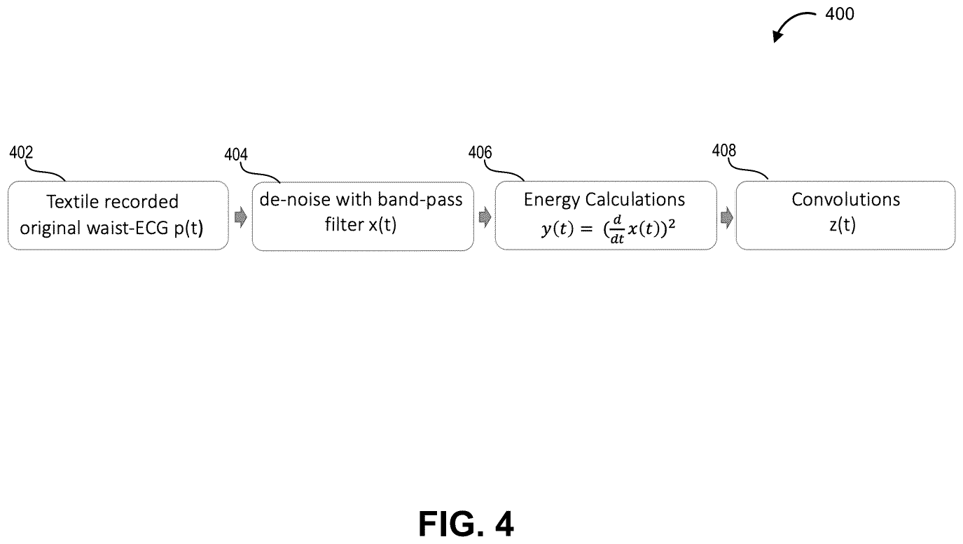

FIG. 4 illustrates another example method 400 of signal pre-processing, according to some embodiments.

After band-pass filters, the ECG signal can be further smoothened to reduce the SNR. An accurate smoothing technique can be developed with Gaussian kernels (the main consideration can be to assign higher weights to R-peaks (to stress) and lower weights to noise (to suppress)). After recording the signal (402), the signal can be pre-processed. This step can consist of 3 sub-steps (1) bandpass filter (404), (2) derivative and energy calculation (406), (3) smoothing with convolution (408). The bandpass filter (404) can reduce high frequency noise and baseline drift (motion artifacts). Derivative and energy calculation (406) and smoothing with convolution (408) can increase the SNR.

Referring back to FIG. 3, Step II. R-peak detections (304): At each R-peak in the pre-processed signal, the distribution of the next R-peak can be estimated as the HDIG with real-time updated model parameters based on the recent R-peak history. A detailed local search can be conducted in a structured searching interval around the HDIG predicted time point for the next R-peak, which passes an adaptive threshold (312) to generate 320.

In some embodiments, the R-peak detection result 320 of the PS method can optionally be compared with the reference (true) R-peaks 322 in the annotation file for training.

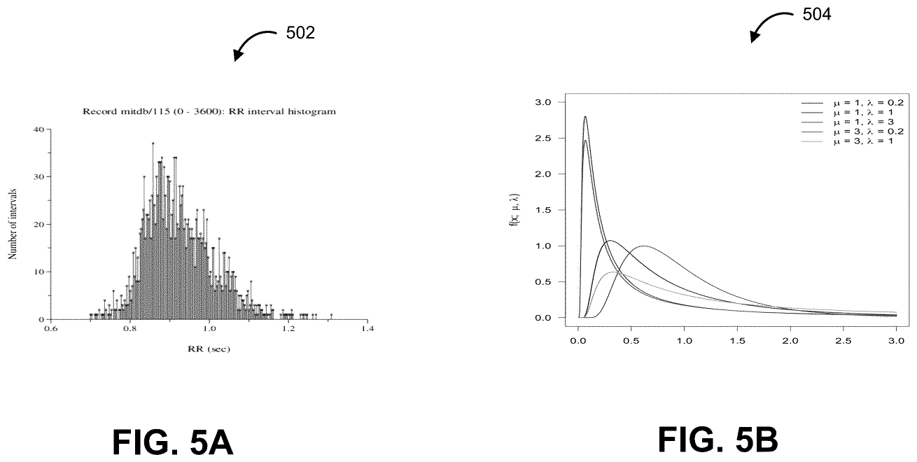

FIG. 5A illustrates a sample distribution of RR intervals in 30 minutes (502), according to some embodiments. FIG. 5B illustrates an Inverse Gaussian distribution (504), according to some embodiments.

The Inverse Gaussian distribution (504) can fit the RR distributions (502) better than normal distribution. Inverse Gaussian distribution may be more precise for a biological system because it starts from 0 (no negative values). HDIG (History Dependent Inverse Gaussian) models can be used to model the distributions of RR intervals. The HDIG model assumes that RR intervals follow an inverse Gaussian distribution and the parameters in the distribution are “history dependent” (meaning that the current RR interval can be, for example, a linear combination of previous RR intervals). A maximum likelihood method can be implemented to find the linear parameters. The basic idea of maximum likelihood can be to predict the RR interval that has the highest probability from the recorded current RR interval.

With the Gaussian kernel convolution technique, the number and amplitude of the noise peaks may be decreased and the SNR may effectively be increased. However, some large motion artifacts can be intrinsic to the raw signal and may not be removed by the pre-processing. The R-peak detection step with the probabilistic search methodology may increase the detection accuracy by searching around the more probable time point. The structured searching interval can effectively avoid the large amplitude motion artifacts and locate the true R-peaks.

### Example Implementations of Systems and Methods for Probabilistic Searching for R-Peak Detection

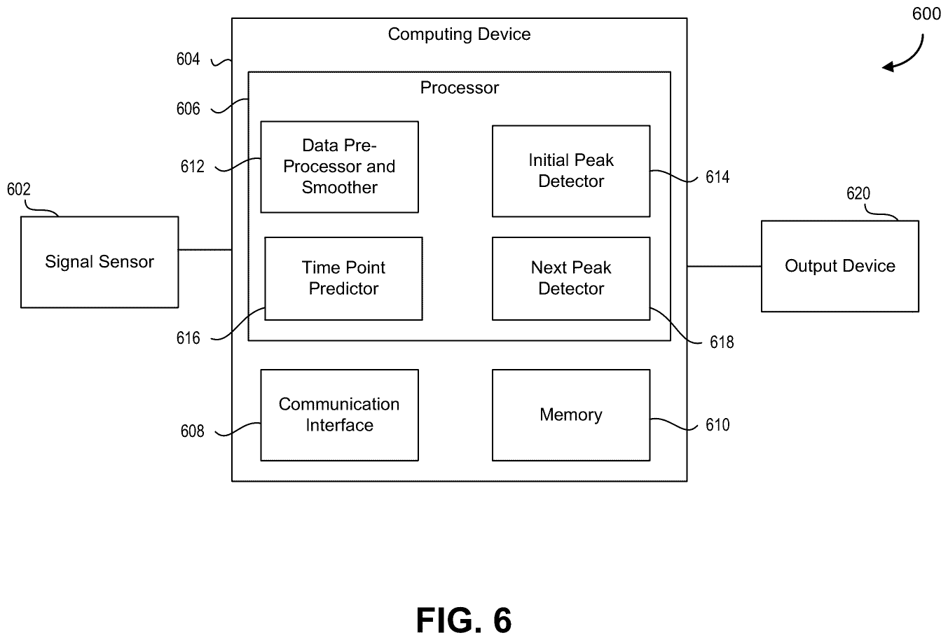

FIG. 6 illustrates an example system 600 for detecting a next peak using probabilistic distribution, according to some embodiments. The system 600 can include a signal sensor 602, a computing device 604, and an output device 620.

The signal sensor 602 may be any sensor configured to sense signals. In some embodiments, the signal sensor 602 can be a bio-signal sensor configured to sense signals arising from a body of a user for, for example, ongoing health monitoring. In some embodiments, the signal sensor 602 may be an electrocardiogram configured to receive electric signals indicative of cardiac activity (e.g., electrical signals from the heart).

The computing device 604 can include, for example, a personal computer, a mobile device executing, for example, an app, and/or a server offering Software as a Service. The computing device 604 can be a single computing device 604 or can be made up of a plurality of computing devices 604 each carrying out one or more operations described herein. The computing device 604 includes a processor 606 configured to carry out various processes, a communication interface 608 for communicating with external and/or third-party devices, and a memory 610 for storing data and/or instructions for carrying out processes.

The processor 606 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof. The processor 606 may be configured with instructions to carry out a probabilistic search for peaks as described herein. In some embodiments, the processor 606 may be configured with a data pre-processor and smoother 612, an initial peak detector 614, a time point predictor 616, and/or a next peak detector 618.

The data pre-processor and smoother 612 may be configured to receive signals from the signal sensor 602 and process and smooth the data. For example, the data pre-processor and smoother 612 may be configured to apply a median filter and bandpass filter, conduct derivative and energy calculations, and smooth with convolution. In some embodiments, smoothing can be carried out using a Gaussian kernel that may increase SNR. The goal may be to reduce noise in the data stream, amplify the signal, reduce the baseline drift, remove high frequency noise, and/or increase the SNR so that the R-peaks are easier to detect. Other pre-processing and smoothing methods may be utilized by data pre-processor and smoother 612. In some embodiments, data pre-processing and smoothing may not be carried out.

In some embodiments, the method may work for any ECG signal containing R-peaks (without pre-processing). In some embodiments, the baseline drift can be removed to make the data features (e.g., peaks) clearer. In some embodiments, the pre-processing step can clean the signal to some extent, and can increase SNR to facilitate the feature detections. This can be particularly helpful for wearable-ECG to increase the R-peak detection accuracy given that wearable-ECG can be noisy. A smoothing method was developed by convolving with a Gaussian kernel which can be effective in increasing SNR.

The initial peak detector 614 may be configured to detect peaks (e.g., R-peaks) in an initial time interval of the pre-processed data stream (e.g., ECG data). The initial peak detector 614 may be configured to detect an initial peak using the methods described herein (e.g., in an iterative manner) or in a manner different from that described herein (e.g., through an alternative methodic manner or through user input).

In some embodiments, initial peak detector 614 uses a different method to detect the very first ˜5 R-peaks to initialize monitoring with some R-peak history. In some embodiments, the choice of the different method can be made at random. In some embodiments, the method of initialization can be PT, any open-source method, or artificial inputs (e.g., fix the first 5 RR intervals to be all 800 ms). Described herein are results that use the open-source Khamis method for detecting the first ˜5 R-peaks, because Khamis may be more robust to noise than the other methods. The methods described herein are robust to the choice of the initializing method and can immediately correct itself. The R-peak detection accuracy may be identical regardless of which initializing method is selected.

The time point predictor 616 may be configured to generate a predicted time point of a first new peak (e.g., an R-peak) by predicting a probabilistic distribution of the new peak following a last peak in the initial time interval (i.e., the peak detected by the initial peak detector 614) with, for example, HDIG distribution. In some embodiments, the distribution parameters can be updated in real-time based on peak history. In some embodiments, the distribution parameters can be updated in real-time based on recent peak history. In some embodiments, other probabilistic distributions may be used.

In some embodiments, the peak detection can be based on its underlying probabilistic distribution. For example, the methods described herein may not be restricted to ECG signal and HDIG distribution. In some embodiments, it may be necessary to incorporate more complex models where the underlying mechanism cannot be characterized by probabilistic distribution solely. For example, in neural systems, the dynamics of a neuron may be affected by complex interplays among neurons, and such mechanisms may not be sufficiently characterized by one form of probabilistic distribution. Thus, neural systems may benefit from more complex models.

The next peak detector 618 may be configured to detect the first new peak (e.g., an R-peak) in the data stream (e.g., ECG data) by searching for the first new peak around the predicted time point of the first new peak in an adaptive searching interval. In some embodiments, the peak with a largest amplitude passing a varying adaptive threshold may be detected as the first new R-peak. In some embodiments the searching interval may be symmetric or asymmetric. In some embodiments, the searching interval may be skewed to, for example, mimic physiological conditions such as an arrhythmia. In some embodiments, the searching interval may expand by, for example, a step. In some embodiments, the system may be configured to lower the adaptive threshold once a first searching interval is exhausted and re-search the same interval with the lower adaptive threshold. In such embodiments, the system may be configured to continue expanding the searching interval in, for example, a stepwise manner if no peak is identified as meeting the lowered adaptive threshold in the original interval.

In some embodiments, once the next peak is detected by next peak detector 618, the system may be configured to detect remaining peaks (e.g., R-peaks) following the first new peak in the data stream (e.g. ECG data) iteratively and in real-time as the data stream is received by submitting the first new peak as a subsequent initial peak to initial peak detector 614 and allowing the method of initial peak detector 614, time point predictor 616, and next peak detector 618 to iterate until, for example, the full data stream is analyzed or until some other criteria is met.

In some embodiments, the system may be configured to further measure an average peak rate by dividing the total number of peaks detected in the data stream by the time length of the data stream. In some embodiments, the system may be configured to measure an average heart rate in the data stream as a total number of detected R-peaks in the data stream divided by a time length of the data stream. In some embodiments, average peak rate may be determined in another way, for example, by determining a recent average peak rate in the data stream (rather than relying on the full data stream).

In some embodiments, the average heart rate can be computed on different time lengths (e.g., 10 s, 20 s, 60 s (bpm). Such averages may not require the kernel method (e.g., convolution with a Gaussian kernel).

Communication interface 608 may enable computing device 604 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these. Communication interface 608 can be configured to communicate with, for example, signal sensor 602 and/or output device 620. Communication interface 608 may enable computing device 604 to interconnect with one or more input devices, such as a keyboard, mouse, camera, touch screen and a microphone, or with one or more output devices 620 such as a display screen and a speaker.

Memory 610 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 610 may store executable instructions for carrying out the processes of data pre-processor and smoother 612, initial peak detector 614, time point predictor 616, next peak detector 618, or other processes carried out by processor 606. In some embodiments, memory 610 may include non-transitory memory for storing the executable instructions for the R-peak detection and heart rate monitoring.

Output device 620 may be configured to output a report or feedback based in part on the peak detection and/or peak rate monitoring carried out by computing device 604. In some embodiments, output device 620 may be external to computing device 604. In some embodiments, output device 620 may be integral or otherwise connected to computing device 604.

According to an aspect there is provided a system 600 for probabilistic search for R-peak detection from electrocardiogram (ECG). The system 600 includes a communication interface 608 to receive a data stream including an ECG signal, a hardware processor 606 programmed with executable instructions for the R-peak detection and heart rate monitoring, non-transitory memory 610 for storing the executable instructions for the R-peak detection, and an output device 620 for transmitting and/or storing the heart rate metrics. The hardware processor 606 pre-processes and smooths the data stream including the ECG signal to reduce noise in the data stream and amplify the ECG signal using the data pre-processor and smoother 612, detects R-peaks in an initial time interval of the pre-processed data stream using initial peak detector 614, generates a predicted time point of a first new R-peak by predicting a probabilistic distribution of the new R-peak following a last R-peak in the initial time interval with History Dependent Inverse Gaussian (HDIG) distribution using time point predictor 616, detects the first new R-peak in the data stream by searching for the first new R-peak around the predicted time point of the first new R-peak in an adaptive searching interval using next peak detector 618, detects remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak using time point predictor 616, and searching in the adaptive searching interval around the predicted time point using next peak detector 618, compute heart rate metrics using the detected R-peaks. Distribution parameters are updated in real-time based on recent R-peak history. The peak with a largest amplitude passing a varying adaptive threshold is detected as the first new R-peak. Peaks with largest amplitudes passing the varying adaptive threshold are detected as the remaining R-peaks. The heart rate metrics are one or more of average heart rate, accuracy, F1-score, sensitivity, precision, and heart rate variability.

In some embodiments, the average heart rate (bpm) can be output from many heart health monitoring systems. The precision measure can be affected by the ability of the algorithm to avoid false alarms (FP). The sensitivity measure can be affected by the ability of the algorithm to reduce the number of missed detections (FN). F1-score can be the harmonic mean of precision and sensitivity, and can often be used as a standardized accuracy measure. All of these heart rate metrics may be helpful to provide as output or as part of further processing.

According to a further aspect, the hardware processor 606 measures the average heart rate in the data stream as a total number of detected R-peaks in the data stream divided by a time length of the data stream.

According to a further aspect, the hardware processor 606 is programmed with executable instructions for continuous heart rate monitoring by continuously receiving the data stream and re-computing the heart rate metrics.

According to a further aspect, the system 600 further includes a band-pass filter to pre-process the data stream to reduce baseline wander and high frequency noise.

According to a further aspect, the hardware processor 606 pre-processes and smooths the data stream using an energy calculator that amplifies the R-peaks in the ECG signal.

According to a further aspect, the hardware processor 606 pre-processes and smooths the data stream using a smoothing function that convolves the data stream with a Gaussian kernel specific to a sampling frequency of the data stream and a type of sensor recording the data stream to further increase the signal-to-noise ratio (SNR).

According to a further aspect, the hardware processor 606 pre-processes and smooths the data stream with a Gaussian kernel by assigning higher weights to R-peaks in the ECG signal and lower weights to noise in the data stream.

According to a further aspect, distribution parameters are updated in real-time based on recent R-peak history with a fast optimization technique such that more recent R-peak history has higher weights than more previous R-peak history.

According to a further aspect, the adaptive searching interval depends on the predicted time point of the first new R-peak.

According to a further aspect, the adaptive searching interval depends on patient specific skew parameters based on an extent of arrhythmia selected from the group consisting of slight arrhythmia, moderate arrhythmia, and serious arrhythmia.

According to a further aspect, the adaptive searching interval is at an interval of increasing length.

According to a further aspect, the varying adaptive threshold is specific to a type of sensor recording the data stream.

According to a further aspect, the adaptive searching interval is generated by training on datasets based on a large amount of ECG data.

According to a further aspect, the system 600 includes one or more electrodes to perform measurements to record the raw ECG signal.

According to a further aspect, the electrodes are dry-electrodes.

According to a further aspect, the electrodes are gel-electrodes.

According to a further aspect, the system 600 includes one or more wearable sensors to perform measurements to record the raw ECG signal.

According to a further aspect, the one or more wearable sensors comprise one or more wearable textile sensors.

According to a further aspect, the HDIG distribution extracts an underlying probabilistic structure of R-peak positions in the data stream based on deterministic morphological features of ECG signals.

According to a further aspect, model parameters for the HDIG distribution are updated in real-time.

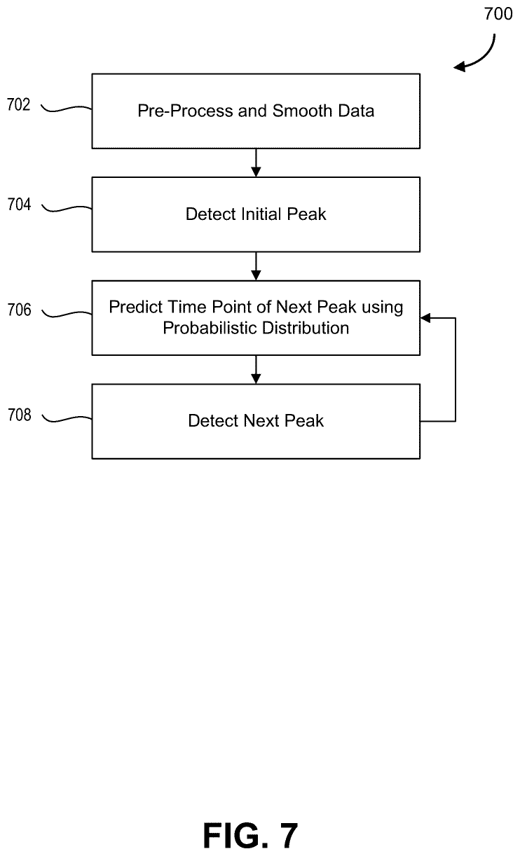

FIG. 7 illustrates an example method 700 for detecting a next peak using probabilistic distribution, according to some embodiments.

The method 700 may include steps for detecting peaks (e.g., R-peaks) within a data stream (e.g., ECG data). The method 700 may include pre-processing and smoothing the data to, for example, reduce noise in the data stream and amplify the signal (702), detecting peaks (e.g., R-peaks) in an initial time interval of the pre-processed data stream (704), generating a predicted time point of a first new peak (e.g., R-peak) by predicting a probabilistic distribution of the new peak following a last peak in the initial time interval with HDIG distribution (706), detecting the first new peak (e.g., R-peak) in the data stream by searching for the first new peak around the predicted time point of the first new peak in an adaptive searching interval (708). In some embodiments, the peak with a largest amplitude passing a varying adaptive threshold is detected as the first new peak. In some embodiments, distribution parameters are updated in real-time based on recent peak history.

In some embodiments the method further includes detecting remaining peaks (e.g., R-peaks) following the first new peak in the data stream iteratively and in real-time as the data stream is received. For each of the remaining peaks, the method may generate a predicted time point of a peak following a most recently detected peak by predicting an HDIG distribution of the peak following the most recently detected peak and search in the adaptive searching interval around the predicted time point.

In some embodiments, the method may further include measuring an average peak rate (e.g., heart rate) in the data stream (e.g., ECG data) as a total number of detected peaks in the data stream divided by a time length of the data stream. In some embodiments, the average peak rate may be calculated in a different averaging manner (e.g., recent real-time averages).

According to an aspect there is provided a method 700 for probabilistic search for R-peak detection from electrocardiogram (ECG). The method 700 includes pre-processing and smoothing a data stream including the ECG signal to reduce noise in the data stream and amplify the ECG signal (block 702), detecting R-peaks in an initial time interval of the pre-processed data stream (block 704), generating a predicted time point of a first new R-peak by predicting a probabilistic distribution of the new R-peak following a last R-peak in the initial time interval with History Dependent Inverse Gaussian (HDIG) distribution (block 706), detecting the first new R-peak in the data stream by searching for the first new R-peak around the predicted time point of the first new R-peak in an adaptive searching interval (block 708), detecting remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak (block 706), and searching in the adaptive searching interval around the predicted time point (block 708), computing heart rate metrics using the detected R-peaks. Distribution parameters are updated in real-time based on recent R-peak history. The peak with a largest amplitude passing a varying adaptive threshold is detected as the first new R-peak. Peaks with largest amplitudes passing the varying adaptive threshold are detected as the remaining R-peaks. The heart rate metrics being one or more of average heart rate, accuracy, F1-score, sensitivity, precision, and heart rate variability.

In some embodiments, the average heart rate (bpm) can be output from many heart health monitoring systems. The precision measure can be affected by the ability of the algorithm to avoid false alarms (FP). The sensitivity measure can be affected by the ability of the algorithm to reduce the number of missed detections (FN). F1-score can be the harmonic mean of precision and sensitivity, and can often be used as a standardized accuracy measure. All of these heart rate metrics may be helpful to provide as output or as part of further processing.

According to a further aspect, the method 700 includes measuring the average heart rate in the data stream as a total number of detected R-peaks in the data stream divided by a time length of the data stream.

According to a further aspect, the method 700 includes continuously processing the data stream and re-computing the heart rate metrics.

According to a further aspect, the preprocessing and smoothing (block 702) includes band-pass filtering the data stream to reduce baseline wander and high frequency noise.

According to a further aspect, the preprocessing and smoothing (block 702) uses an energy calculator that amplifies the R-peaks in the ECG signal.

According to a further aspect, the preprocessing and smoothing (block 702) uses a smoothing function that convolves the data stream with a Gaussian kernel specific to a sampling frequency of the data stream and a type of sensor recording the data stream to further increase the signal-to-noise ratio (SNR).

According to a further aspect, the preprocessing and smoothing (block 702) uses a Gaussian kernel by assigning higher weights to R-peaks in the ECG signal and lower weights to noise in the data stream.

According to a further aspect, distribution parameters are updated in real-time based on recent R-peak history with a fast optimization technique such that more recent R-peak history has higher weights than more previous R-peak history.

According to a further aspect, the adaptive searching interval depends on the predicted time point of the first new R-peak.

According to a further aspect, the adaptive searching interval depends on patient specific skew parameters based on an extent of arrhythmia selected from the group consisting of slight arrhythmia, moderate arrhythmia, and serious arrhythmia.

According to a further aspect, the adaptive searching interval is at an interval of increasing length.

According to a further aspect, the varying adaptive threshold is specific to a type of sensor recording the data stream.

According to a further aspect, the adaptive searching interval is generated by training on datasets based on a large amount of ECG data.

According to a further aspect, the data stream includes input from one or more electrodes performing measurements to record the raw ECG signal.

According to a further aspect, the electrodes are dry-electrodes.

According to a further aspect, the electrodes are gel-electrodes.

According to a further aspect, the data stream includes input from one or more wearable sensors performing measurements to record the raw ECG signal.

According to a further aspect, the one or more wearable sensors include one or more wearable textile sensors.

According to a further aspect, the HDIG distribution extracts an underlying probabilistic structure of R-peak positions in the data stream based on deterministic morphological features of ECG signals.

According to a further aspect, model parameters for the HDIG distribution are updated in real-time.

According to an aspect there is provided a non-transitory computer readable medium with instructions for R-peak detection from electrocardiogram (ECG) stored thereon. When executed by a hardware processor cause the processor to pre-process and smooth a data stream including the ECG signal to reduce noise in the data stream and amplify the ECG signal (block 702), detect R-peaks in an initial time interval of the pre-processed data stream (block 704), generate a predicted time point of a first new R-peak by predicting a probabilistic distribution of the new R-peak following a last R-peak in the initial time interval with History Dependent Inverse Gaussian (HDIG) distribution (block 706), detect the first new R-peak in the data stream by searching for the first new R-peak around the predicted time point of the first new R-peak in an adaptive searching interval (block 708), detect remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak (block 706), and searching in the adaptive searching interval around the predicted time point (block 708), compute heart rate metrics using the detected R-peaks, the heart rate metrics being one or more of average heart rate, accuracy, F1-score, sensitivity, precision, and heart rate variability. Distribution parameters are updated in real-time based on recent R-peak history. The peak with a largest amplitude passing a varying adaptive threshold is detected as the first new R-peak. Peaks with largest amplitudes passing the varying adaptive threshold are detected as the remaining R-peaks.

In some embodiments, the average heart rate (bpm) can be output from many heart health monitoring systems. The precision measure can be affected by the ability of the algorithm to avoid false alarms (FP). The sensitivity measure can be affected by the ability of the algorithm to reduce the number of missed detections (FN). F1-score can be the harmonic mean of precision and sensitivity, and can often be used as a standardized accuracy measure. All of these heart rate metrics may be helpful to provide as output or as part of further processing.

According to a further aspect, the instructions cause the processor to measure the average heart rate in the data stream as a total number of detected R-peaks in the data stream divided by a time length of the data stream.

According to a further aspect, the instructions cause the processor to continuously process the data stream and re-compute the heart rate metrics.

According to a further aspect, the preprocessing and smoothing (block 702) comprises band-pass filtering the data stream to reduce baseline wander and high frequency noise.

According to a further aspect, the preprocessing and smoothing (block 702) uses an energy calculator that amplifies the R-peaks in the ECG signal.

According to a further aspect, the preprocessing and smoothing (block 702) uses a smoothing function that convolves the data stream with a Gaussian kernel specific to a sampling frequency of the data stream and a type of sensor recording the data stream to further increase the signal-to-noise ratio (SNR).

According to a further aspect, the preprocessing and smoothing (block 702) uses a Gaussian kernel by assigning higher weights to R-peaks in the ECG signal and lower weights to noise in the data stream.

According to a further aspect, distribution parameters are updated in real-time based on recent R-peak history with a fast optimization technique such that more recent R-peak history has higher weights than more previous R-peak history.

According to a further aspect, the adaptive searching interval depends on the predicted time point of the first new R-peak.

According to a further aspect, the adaptive searching interval depends on patient specific skew parameters based on an extent of arrhythmia selected from the group consisting of slight arrhythmia, moderate arrhythmia, and serious arrhythmia.

According to a further aspect, the adaptive searching interval is at an interval of increasing length.

According to a further aspect, the varying adaptive threshold is specific to a type of sensor recording the data stream.

According to a further aspect, the adaptive searching interval is generated by training on datasets based on a large amount of ECG data.

According to a further aspect, the data stream includes input from one or more electrodes performing measurements to record the raw ECG signal.

According to a further aspect, the electrodes are dry-electrodes.

According to a further aspect, the electrodes are gel-electrodes.

According to a further aspect, the data stream includes input from one or more wearable sensors performing measurements to record the raw ECG signal.

According to a further aspect, the one or more wearable sensors comprise one or more wearable textile sensors.

According to a further aspect, the HDIG distribution extracts an underlying probabilistic structure of R-peak positions in the data stream based on deterministic morphological features of ECG signals.

According to a further aspect, model parameters for the HDIG distribution are updated in real-time.

### Detailed Exemplary Probabilistic Search ECG R-Peak Detection Method

In greater detail, an exemplary Probabilistic Search (PS) method can consist of 2 main steps: signal pre-processing and R-peak detection. This method may be effective for ECG signals from all sources. These signals may include waist-ECG, wrist-ECG, and chest-ECG, recorded by either gel-electrodes (e.g., MITDB) or dry electrode (e.g., textile). In the PS method, the optimal concrete parameters vary and may be based on the origin of the ECG signals. The PS method can be real-time in the sense that it can finish processing 30-minute ECG data in, for example, ˜15 seconds.

### I. Signal Pre-Processing

This step can consist of 3 sub-steps: (1) median filter and bandpass filter; (2) derivative and energy calculations; and (3) smoothing with convolution. A smoothing technique can be developed with a Gaussian kernel that efficiently increases the SNR. The goal may be to reduce the baseline drift, remove high frequency noise, and increase the SNR so that the R-peaks are easier to detect. The details of each sub-step are stated as follows.

### I. (1). Median Filter and Bandpass Filter

First, the linear trend can be removed from the original ECG by reducing the optimal straight-line fit from the signal. After this, a median filter can be applied with a window width of, for example, 0.5 s to reduce slow baseline wandering. Then, a zero-phase digital bandpass filter can be applied. A high pass filter with a cutoff frequency of, for example, 0.5 Hz and a low pass filter with cutoff frequency of, for example, 20 Hz can be used. The high pass filter can be used to remove the baseline wander and the low pass filter can be used to remove high frequency noises. The transfer function coefficients [a, b] of a 7th-order digital Butterworth filter (for example, using MATLAB function “butter”) with the low and high cutoff frequencies (i.e. [0.5, 20]) can be found. Then a zero-phase digital filtering (for example, using MATLAB function “filtfilt”) can be used with filter order [a, b]. The resulting signal can be denoted as h(t).

### I. (2). Derivative and Energy Calculation

First, a finite impulse response derivative filter to h(t) can be applied (result of sub-step I. (1)). The rational transfer function

$$Y ( z ) = \frac{fs}{2} ( 1 - {z}^{- 2} )$$

(for example, using MATLAB function “filter”) can be used. fs can be the sampling frequency. The resulting signal can be denoted as x(t).

To enhance the representation of the R-peaks, the energy of x(t) can be calculated.

$$E n [ x ( t ) ] = {( \frac{d x ( t )}{d t} )}^{2} \qquad ( 2 )$$

where En[⋅] can denote the time-varying energy of the derivative estimate x(t). The goal can be to amplify the effects of instances with abrupt changes (i.e., increase SNR).

### I. (3). Convolution (Smoothing)

To eliminate the fluctuations surrounding the local peaks in the calculated energy (by (2)), En[x(t)] can be filtered by a narrow Gaussian kernel N(0, 1). The interval I=[−5, 5] (length=10) can be considered to convolve. “Resolution” means the number of sub-divisions (m) of [−5, 5]. For example, the distance between adjacent sub-division points can be set as dt=0.15, then

$$m = \frac{10}{d t} .$$

The appropriate resolution may depend on the origin of the ECG signal.

Suppose the length of signal can be p, then the explicit computational formula of the convoluted signal Z(t)=En[x(t)] *N(μ, ρ²) (* means convolution) can be given by the following:

$$Z ( t ) = {\sum }_{i + j = t} E n [ x ( i ) ] \times N ( 0 , - 5 σ + j * dt ) , t = 1 , 2 , … m + p ; i \geq 1 , j \geq 0 \qquad ( 3 )$$

In practice, p>>m. Then,

$$floor ( \frac{m}{2} )$$

can be truncated sampling points from the front and bottom of Z(t), respectively. This truncation can serve two purposes: (1) the resulting signal can have the same length as En[x(t)], and (2) the location of peaks in the convoluted signal can be adjusted to the correct location as in the pre-convolution signal En[x(t)].

Each resolution m corresponds to a convolution length of the textile-ECG signal, and the length of the convolution window (WL) can be given by:

$$W L = m * \frac{1000}{f s} \qquad ( 4 )$$

where fs can be the sampling frequency. For example, if fs=200 Hz,

$$m = \frac{10}{d t}$$

and dt=0.15, then the length of convolution window (WL) may be given by:

$$W L = m * 5 ms = \frac{10}{0.15} * 5 = 333 ms .$$

WL for ECG recorded with textile sensors can be slightly smaller than with gel-electrodes. The purpose can be to reduce the deviations caused by large artifacts in textile signals. This step can improve R-peak detection by smoothing the fluctuations near the R-peak of the energy signal. The SNR may further be increased.

Calculating linear local average can be the smoothing approach. At each t, such approach replaces En[x(t)] by the average value in a window (t−d, t+d) surrounding t, and all values in the window have equal weights. The approach focused on herein (convolution with N(μ, σ²)) may be different because higher weight can be assigned to the center value, and the adjacent values have decaying weights (e.g., the bell shape of N(μ, σ²)). Such characterization can be advantageous in amplifying signals and suppressing noise (i.e., increasing the SNR), because the center value always weights higher. In many samples, convolution with N(μ, σ²) can improve the SNR, thus R-peak detection accuracies can be improved.

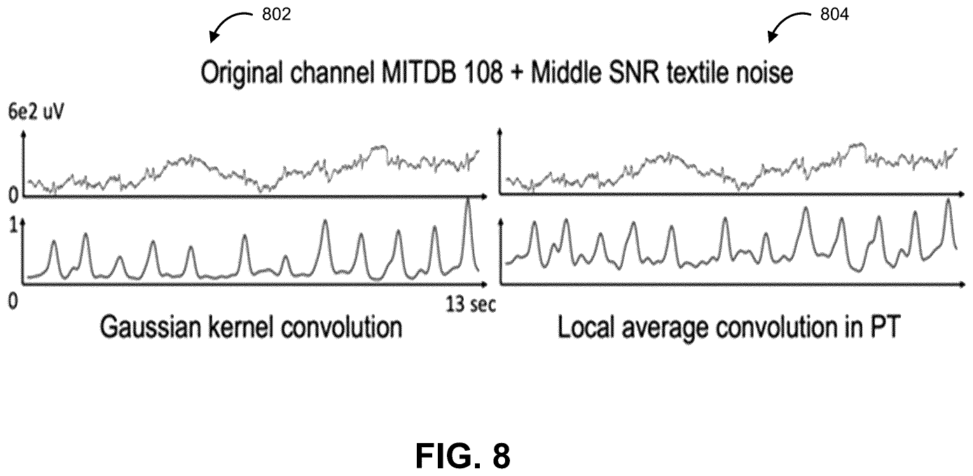

FIG. 8 illustrates a comparison in 13 s segments of the recording “MITDB recording 108+Middle SNR textile noise” in the generated MITDB dataset (by adding textile noise to the original recordings) between the local average method (as in PT; 802) and smoothing method described herein (804), according to some embodiments. The original ECG signals follow exactly the same processing sub-steps I. (1) and I. (2), as introduced above. The only difference is in sub-step I.(3): smoothing. Convolution with a Gaussian kernel can decrease both amplitudes and variance of the noise. This can be an important method to improve signal processing quality.

### II. R-Peak Detection

An iterative probabilistic approach based on prediction and update strategies can be used to detect the peaks of Z(t). This step can consist of 2 sub-steps: (1) prediction; and (2) search. An optimal search method can be used to detect R-peaks based on History Dependent Inverse Gaussian (HDIG) point process model of heartbeat intervals, adaptive searching intervals and adaptive thresholds.

### II. (1). Prediction

The goal of this sub-step can be to predict the time point around which R-peaks are the most probable to happen. Estimated RR intervals can first be calculated based on HDIG, then the associated likelihood function can be optimized with a trust region method.

### II. (1). (i). HDIG Estimate of RR Intervals

RR intervals may follow the Inverse Gaussian distribution, and the parameters in the distribution may depend on the recent R-peak history. At each R-peak, the mean of the distribution may provide the time point around which the next R-peak will most probably happen.

Given any R-peak index uₙ, the RR interval initiating at uₙ can be assumed to follow HDIG. It may be predicted as the mean of HDIG distribution at uₙ. All the HDIG predictions can be considered within a prior β(s) interval (window) prior to uₙ, and the local maximum likelihood method can be used to compute the optimal HDIG parameters.

To be more specific, suppose, for example, the R-peak points in Z(t) (the processed ECG signal after pre-processing in Step I) in β(s) prior to uₙ is given by {u₁, u₂, . . . , uₙ}, then the HDIG probability density function at uₖ (2≤k≤n) can be defined as:

$$f ( t \vert {H}_{{u}_{k}} , θ ) = {[ \frac{{θ}_{p + 1}}{2 {π ( t - {u}_{k} )}^{3}} ]}^{\frac{1}{2}} * exp \{ - \frac{1}{2} * \frac{{{θ}_{p + 1} [ t - {u}_{k} - μ ( {H}_{{u}_{k}} , θ ) ]}^{2}}{{μ ( {H}_{{u}_{k}} , θ )}^{2} ( t - {u}_{k} )} \} \qquad ( 5 )$$

where t>uₖ can be any time point, p can be the number of history RR intervals considered at uₖ. Hᵤ_{ₖ}=(uₖ, wₖ, wₖ₋₁, . . . , wₖ₋ₚ₊₁) can be the vector that contains current time point and the history information. wₖ=uₖ−uₖ₋₁ is the (k−1)^{th} RR interval. θ=(θ₀, θ₁, . . . , θₚ, θₚ₊₁) can be the time-varying parameter vector. μ(Hᵤ_{ₖ}, θ)=θ₀+Σⱼ₌₁^{p}θⱼwₖ₋ⱼ₊₁ can be the mean, θ₀ can be the initial estimate of RR interval and θₚ₊₁>0 can be the shape parameter of the HDIG distribution. To initialize the predictions, the R-peaks in the initial 5 seconds of Z(t) can be detected with an initializing method. The choice of this initializing method may be permissive, because the PS method can be robust to the detection errors in the short initial 5 seconds. For the example described herein the Khamis method (open source) was used.
After defining HDIG density, the next step can be to estimate θ so that the estimate RR

interval initiating at uₙ can be obtained, i.e.

$${\hat{R R}}_{n} = - {u}_{n} = μ ( {H}_{{u}_{n}} , θ ) \qquad ( 6 )$$

A local maximum likelihood method can be implemented to estimate θ. In the prior β to uₙ, the likelihood function can be defined as:

$$L ( β , {u}_{n} , θ ) = {\prod }_{i = 1}^{n} q ( {u}_{n} - {u}_{i} ) f ( {u}_{i + 1} \vert {H}_{i} , θ ) \qquad ( 7 )$$

$$where$$

$$q ( {u}_{n} - s ) = exp ( - α ( {u}_{n} - s ) ) , α \geq 0 , s < {u}_{n} \qquad ( 8 )$$

can be an exponential decay weight function. This can be utilized because the RR intervals closer to uₙ (i.e. the more recent events) may have more influence on the current state. a can be the weight constant, s<uₙ can be any previous time point. The increase of a may decrease the weight of previous events in the local likelihood function.

Finally, the more computationally effective log-likelihood form can be written as:

$$l ( β , {u}_{n} , θ ) = log ( L ( β , {u}_{n} , θ ) ) = {\sum }_{i = 1}^{n} q ( {u}_{n} - {u}_{i} ) log ( f ( {u}_{i + 1} \vert {H}_{i} , θ ) ) \qquad ( 9 )$$

Then (9) can be maximized with a trust region method (explained below in part II. (1).(ii)) to obtain θ. Thus by (6), μ(Hᵤ_{ₙ}, θ) can be the predicted RR interval initiating at uₙ. By the definition of HDIG, around uₙ+μ(Hᵤ_{ₙ}, θ) the next R-peak can be the most probable to happen. Then combined with the next sub-step, an optimal search process can be completed.

### II. (1). (ii). Likelihood Function Optimization

The log-likelihood function (9) can be maximized with a trust region method instead of a line search method like gradient descent. This can reduce the number of iteration steps and increase the computational speed because computing history functions can be computationally expensive. The PS method can finish processing 30-minute ECG data in, for example, ˜ 15 seconds. Thus, the PS method can detect R-peaks in real-time.

The objective function (9) can be denoted by l(θ) since θ is the variable, the gradient and Hessian of l(θ) can be denoted by g(θ) and H(θ), respectively. Then the 2nd order Taylor expansion of l(θ) can be given by:

$$l ( θ + s ) = l ( θ ) + g ( θ ) \cdot s + \frac{1}{2} {s}^{T} H ( θ ) s + o ( {‖ s ‖}^{2} ) \qquad ( 10 )$$

where l: [symbol] → [symbol] ; g(θ), θ, s∈ [symbol] ; H(θ)∈ [symbol] × [symbol] and ∥s∥ can be the L² norm of s. p can be the number of history RR intervals considered at each time point.

The goal here may be to minimize l(θ) after several iterations, thus for each step,

$$l ( θ + s ) - l ( θ ) \approx g ( θ ) \cdot s + \frac{1}{2} {s}^{T} H ( θ ) s$$

should be as small as possible. Thus, the s that minimizes the following equation is sought:

$$q ( s ) := g ( θ ) \cdot s + \frac{1}{2} {s}^{T} H ( θ ) s , ‖ s ‖ \leq Δ \qquad ( 11 )$$

Δ can be called the “trust region radius” and can be the maximal distance allowed from the previous position. The PS method may choose ∥s∥=Δ, so Δ can also mean “step size”. Thus, the number of iterations in the trust region method can be small by choosing reasonably large Δ (e.g., Δ=∥−H(θ)⁻¹g(θ)∥), and the direction of s can be fitted to Δ. This may provide advantages over the gradient descent methods, where the gradient direction can only move along with much smaller step sizes.

The global minimizer (the Newton point) of q(s) can be obtained by solving q′(s)=0⇒s=−H⁻¹g. This solution may not satisfy the trust region ∥s∥≤Δ. The exact minimizer to (11) can be given by:

$$( H + ∝ I ) s = - g ⇒ s = - {( H + ∝ I )}^{- 1} g , and ∝ staisfies ‖ - {( H + ∝ I )}^{- 1} g ‖ = Δ \qquad ( 12 )$$

where ∝ can be the uniquely determined scalar and I can be the identity matrix. In practice, ∝ can be simulated to make ∥−(H+∝I)⁻¹∝ close to Δ. However, this approach may be computationally expensive because the factorization of H+∝I may need to computed in each simulation to compute the matrix inverse. Thus in (11), the minimizer s can be searched for directly in some subspace of {s|∥s∥≤Δ}. It can be numerically shown that searching for s in a two-dimensional subspace generated by {g, −H⁻¹g} (i.e., the gradient direction and the Newton point) may be as accurate as, but much more efficient than, searching the whole space {s|∥s∥≤Δ}. Thus s in span{g, −H⁻¹g} can be chosen with ∥s∥=Δ.

H⁻¹ can be computed with the Conjugate Gradients method, which can give an accurate approximation when H is positive definite. When H is not positive definite, a Preconditioned Conjugate Gradients (PCG) method can be used as follows:

$$\\ \begin{matrix}Hs = - g {E}^{{}_{} - 1} {H ( {E}^{{}_{} - 1} )}^{T} \tilde{s} = - {E}^{{}_{} - 1} g , where \tilde{s} = {E}^{{}_{} T} s ; so s = {( {E}^{{}_{} - 1} )}^{T} \tilde{s} & ( 13 )\end{matrix}$$

M=EE^{T} can be called “preconditioner” and should be symmetric positive-definite and fixed in all iterations. PCG can predict s with a big negative curvature, which can be accurate if cond(E) (matrix condition number) is not too large (e.g., <1e3 or 1e4). Matrix condition number can be close to ∞ when the matrix approaches singularity (determinant→0).

### II. (2). Search

The search for uₙ₊₁ (the next R-peak) can be performed based on adaptive intervals centered around uₙ+μ(Hᵤ_{ₙ}, θ). In the searching intervals, appropriate adaptive thresholds can be implemented and the maximal peak chosen above the threshold as ûₙ₊₁.

### II. (2). (i). Adaptive Searching Intervals

The initial searching interval can be defined as:

$$\\ \begin{matrix}{I}_{ini} = {I}_{0} = ( {u}_{n} + max \{ μ ( {H}_{{u}_{n}} , θ ) - {a}_{n} , r \} , {u}_{n} + μ ( {H}_{{u}_{n}} , θ ) + {a}_{n} ) where & ( 14 )\end{matrix}$$

$$\\ \begin{matrix}{a}_{n} = m * \frac{1}{q} {\sum }_{i = 1}^{q} {w}_{n - i + 1} = m * average of the most recent q RR intervals & ( 15 )\end{matrix}$$

wₖ=uₖ−uₖ₋₁ can be the (k−1)th RR interval, m can be around, for example, 0.2 and can be determined by data. From (15) we can see that Iᵢₙᵢ can adapt to the current data.

The purpose of max{μ(Hᵤ_{ₙ}, θ)−aₙ,r} can be to restrict a refractory period (denoted by r in m-sec) before the next R-peak. In practice, r≥100 ms can be chosen. The fastest human heart rate reported to date in a tachyarrhythmia was 480 beats per minute (corresponding to an RR interval of 125 ms). For some patients, heart rate can reach 600 bpm (RR interval of 100 ms) and last for 20 s. So, the minimum RR interval that can be allowed may be 100 ms, the peaks within 100 ms may mostly be artifacts. These may be the physiological considerations for the choice of r.

If the satisfying peak in Iᵢₙᵢ cannot be found the search can be done on a larger searching interval with step-increment length b, until one can be found. To be specific, the (k+1)^{th} searching interval can be given by:

$$\\ \begin{matrix}{I}_{k} = ( {u}_{n} + max \{ μ ( {H}_{{u}_{n}} , θ ) - {a}_{n} - kb , r \} , {u}_{n} + μ ( {H}_{{u}_{n}} , θ ) + {a}_{n} + kb ) , k \geq 0 & ( 16 )\end{matrix}$$

The increase of searching interval can end if max{μ(Hᵤ_{ₙ}, θ)−aₙ−kb, r}=r, i.e.

$$\\ \begin{matrix}{I}_{max} = ( {u}_{n} + r , {u}_{n} + μ ( {H}_{{u}_{n}} , θ ) + {a}_{n} + {k}_{max} b ) & ( 17 )\end{matrix}$$

During the increasing process of searching interval length, if a satisfying peak cannot be found in an intermediate interval:

$$\\ \begin{matrix}{I}_{c} = ( {u}_{n} + max \{ μ ( {H}_{{u}_{n}} , θ ) - {a}_{n} - c , r \} , {u}_{n} + μ ( {H}_{{u}_{n}} , θ ) + {a}_{n} + c ) & ( 18 )\end{matrix}$$

the threshold can be decreased in the further searches. The length of I_{c} (i.e., 2(aₙ+c)) can also be adjusted according to real data.

Bigeminy or trigeminy in RR interval distributions can be common features in arrhythmia. When analyzing arrhythmia data, the detection accuracy may be increased by applying a skewed searching interval. The step-wise searching interval in (16) can be skewed to the left with a skew parameter τ (τ≥1). The purpose can be to compensate for the possible false negatives when RR intervals suddenly decrease. The amplitude of τ can depend on the extent of arrhythmia. The skewed searching interval can be given by:

$$\\ \begin{matrix}{I}_{k}^{{}_{} τ} = ( τ * [ {u}_{n} + max \{ μ ( {H}_{{u}_{n}} , θ ) - {a}_{n} - kb , r \} ] , {u}_{n} + μ ( {H}_{{u}_{n}} , θ ) + {a}_{n} + kb ) , & ( 19 )\end{matrix}$$

$$\\ k \geq 0$$

In practice, using 3 levels of skew can improve the R-peak prediction accuracy. The levels can be defined as (1) healthy/slight arrhythmia; (2) moderate arrhythmia; and (3) serious arrhythmia. The skew parameter τ can increase as the arrhythmia becomes more serious.

Such skewed searching interval (τ>1) may reduce false negatives and may include only a few more false positives, compared with a symmetric searching interval (τ=1). This may be because we are still near the most probable position for R-peaks, although the searching interval may be skewed.

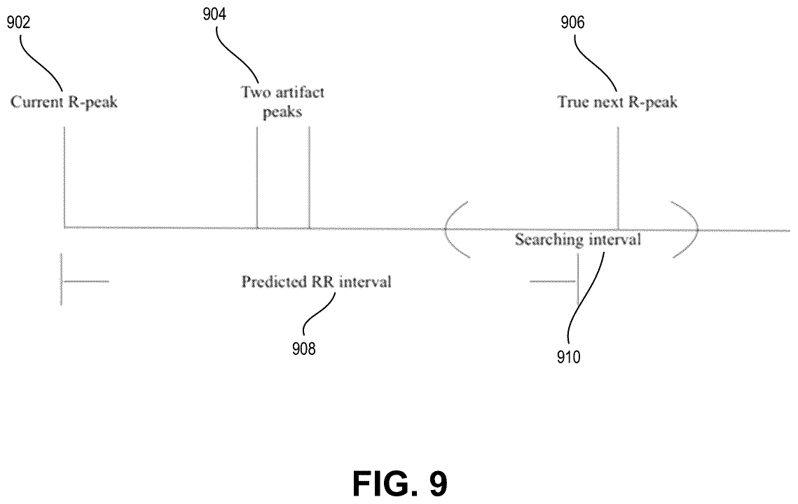

FIG. 9 illustrates a Probabilistic Search (PS) method with a simplified searching interval, according to some embodiments.

In the example PS method, the position of the current R-peak 902 is measured. The predicted RR interval 908 can be predicted based on past RR intervals and used to set the initial searching interval 910 centered on where the predicted RR interval centers the next true R-peak 906. The RR interval can be predicted by the HDIG distribution. Using this method, the artifact peaks 904 can be avoided.

At each R-peak point uₙ, after the [symbol] is obtained, the next R-peak is searched for in an interval centered at uₙ+ [symbol] , around which the next R-peak may be the most probable to happen (thus called “optimal search”). Based on the extent of arrhythmia, the searching interval can be symmetric or skewed, but it is often far from the artifact peaks because the search is around the place where R-peaks are the most probable to happen. The PS method can avoid false positive peaks by searching in the correct place (predicted probabilistically) with the detailed adaptive searching intervals.

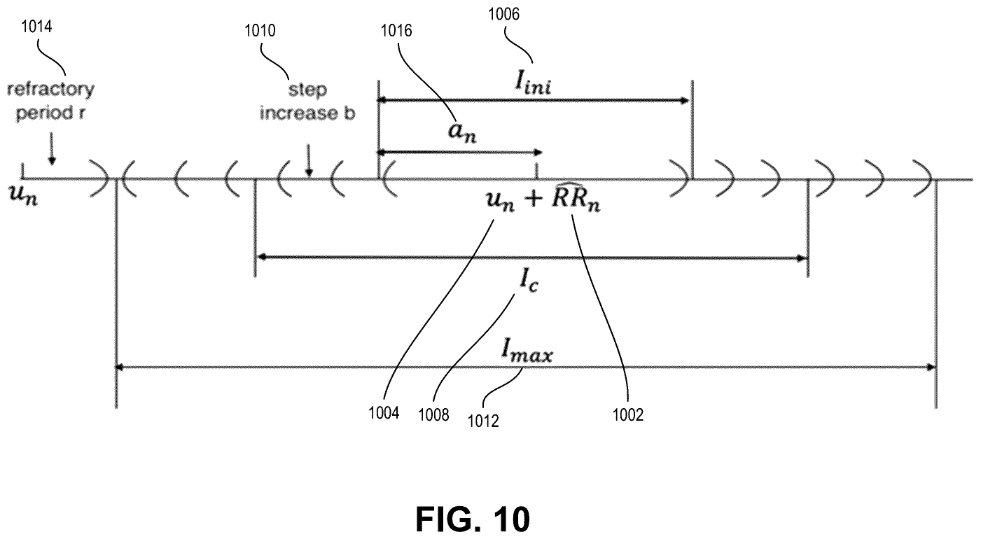

FIG. 10 illustrates a construction of the searching intervals used in the Probabilistic Search (PS) method, according to some embodiments.

In FIG. 10, the predicted RR interval 1002 is [symbol] =μ(Hᵤ_{ₙ}, θ) (see Equation (6)). This interval 1002 added to the prior R-peak 1004 (not shown but provided as a sum) can determine the predicted location of the next R-peak which defines the searching interval Iᵢₙᵢ 1006. aₙ 1016 can represent the history (e.g., the average of the most recent RR intervals, see e.g., Equation (15)). The original adaptive threshold is applied from Iᵢₙᵢ 1006 until I_{c} 1008 in step increases 1010. If no satisfying R-peak is found, then a search-back in I_{c} 1008 is conducted with a decreased threshold. If no satisfying R-peak is found, then this decreased threshold is maintained, and the searching interval increased until Iₘₐₓ 1012. A skew coefficient τ can be applied to the searching intervals (1006, 1008, and/or 1012) for, for example, patients with arrhythmia, if necessary. The refractory period 1014 can be where the next R-peak is not expected because it is too close to the prior R-peak 1004 (and thus is physiologically unlikely if not impossible to present in that period).

R-peak detections can be performed in the searching intervals. The selected R-peak may need to pass an adaptive threshold, and the details are shown below in part II. (2).(ii).

### II. (2). (ii). Adaptive Thresholds

The method of choosing the predicted R-peak from a searching interval can be by picking the maximal peak passing a certain threshold. This threshold can be adaptive, in the sense that its amplitude can be time-varying depending on the running amplitudes of both noise peaks and R-peaks. After the detection of the next R-peak, the next R-peak is updated as the current R-peak and the whole process continues until the analysis is done. To increase computational efficiency, a linear iterative method can be used instead of more complex functions. A two-threshold updating method improved from PT can be applied. Improvements can be made to suggest a method that may be suitable for all sources of ECG signals, recorded by either dry electrode (e.g., textile waist-ECG) or gel-electrode (e.g., MITDB).

One set of thresholds (denoted by TH1) can be designed for the first searches, the other set of thresholds (denoted by TH2) can be designed for further searches. As indicated in section II. (2).(i), “the first searches” may mean searching with TH1 in the increasing searching intervals starting from Iᵢₙᵢ, and the process ends if a satisfying peak can be found. If any peak passing TH1 cannot be found in I_{c} (see (18)), then TH2<TH1 can be applied for a search back in I_{c}. If the satisfying peak still cannot be found, TH2 can remain and the searching interval length can increase until Iₘₐₓ. The maximal peak passing TH2 can be picked as the next R-peak.

The progressing evaluations of TH1 and TH2 can be based on the running estimates of R-peaks (denoted by RPK) and noise peaks (denoted by NPK). RPK (resp. NPK) can be updated after detecting each R-peak (resp. noise peak). So RPK (resp. NPK) can summarize R-peak (resp. noise peak) amplitude information from the starting time point to the current time point.

After the detection of each R-peak, all peaks between the previous R-peak and the current R-peak can be considered as noise peaks and they can be used to update NPK. The amplitude of a certain peak can be denoted by “PEAK”. After RPK and NPK can be updated, TH1 and TH2 can be updated accordingly. The detailed updating formulas in the first searches can be given by:

$$\\ \begin{matrix}RPK = 0.125 * PEAK + 0.875 * RPK , if PEAK is an R - peak & ( 20 )\end{matrix}$$

$$\\ \begin{matrix}NPK = 0.125 * PEAK + 0.875 * NPK , if PEAK is a noise peak & ( 21 )\end{matrix}$$

$$\\ \begin{matrix}TH 1 = wt * RPK + ( 1 - wt ) * NPK & ( 22 )\end{matrix}$$

$$\\ \begin{matrix}TH 2 = α * TH 1 & ( 23 )\end{matrix}$$

The weighting coefficient wt in (22) can be the weight of RPK in TH1. This number can be a variable based on the origin of data. For example, textile recorded signals may have more variations in R-peak amplitudes, thus wt can be smaller to reduce false negatives caused by large R-peaks. α<1 can be the proportion of TH1 for further searches. Usually,

$$\\ α = \frac{1}{2}$$

can be appropriate, and it can be smaller to compensate for smaller R-peaks.

The updating formulas can be slightly different for the further searches with TH2.

$$\\ \begin{matrix}RPK = 0.25 * PEAK + 0.75 * RPK , if PEAK is an R - peak & ( 24 )\end{matrix}$$

$$\\ \begin{matrix}NPK = 0.125 * PEAK + 0.875 * NPK , if PEAK is a noise peak & ( 25 )\end{matrix}$$

$$\\ \begin{matrix}TH 1 = wt * RPK + ( 1 - wt ) * NPK & ( 26 )\end{matrix}$$

$$\\ \begin{matrix}TH 2 = α * TH 1 & ( 27 )\end{matrix}$$

The purpose of the increasing weight of PEAK in RPK can be to stress the current R-peak. Since TH2 can be applied, a satisfying R-peak may not be found in the first searches with TH1. This means that there may be a big difference in amplitudes of the current R-peak. Such may indicate the potential sudden change of current physiological conditions, so it may be stressed in a real-time method.

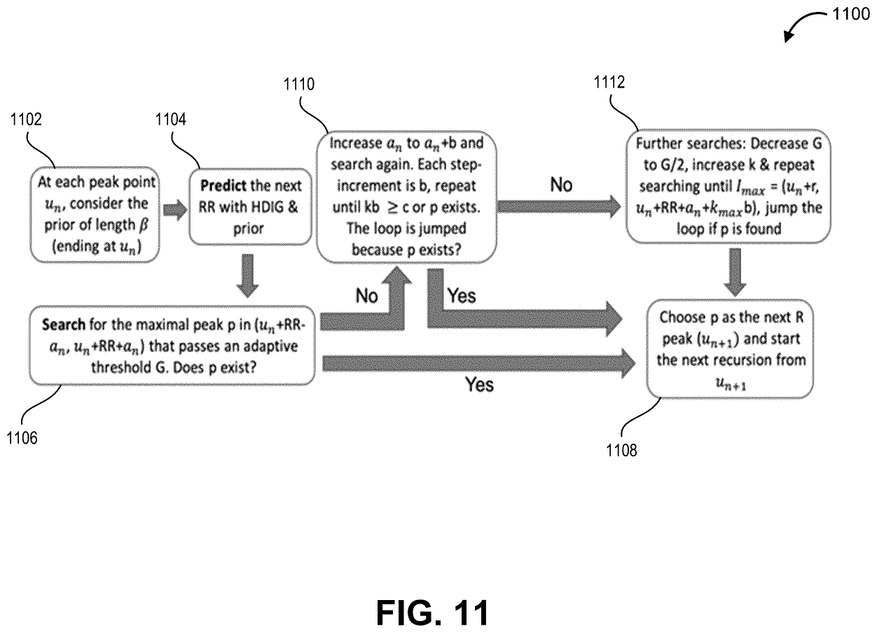

FIG. 11 illustrates an example method 1100 that summaries the R-peak detection step of the Probabilistic (PS) method, according to some embodiments.

At each peak point, uₙ, the prior length of β (ending at uₙ) is considered (1102). The next RR interval can be predicted with HDIG and the prior RR intervals (1104). The maximal peak p is searched in uₙ+ [symbol] −aₙ to uₙ+ [symbol] +aₙ that passes an adaptive threshold G (1106). If an R-peak is found, then that R-peak is used as uₙ₊₁ and the next recursion initiates therefrom (1108). If no peak is found at 1106, then aₙ can be increased to aₙ+b and a subsequent search can be conducted (1110). The increments can be of b and repeated k times until kb≥c or a peak is found. If a peak is found, then the method 1100 advances to block 1108. If no peak is found, then the adaptive threshold G can be decreased to, for example G/2, k can be increased and searching can be repeated until Iₘₐₓ=(uₙ+r, uₙ+ [symbol] +aₙ+kₘₐₓb) (1112). If a peak is then found, then the method 1100 advances to block 1108.

### Exemplary Experimental Results

The following provides results from an exemplary embodiment of the systems and methods described herein. The details of the following discussion in no way limit the full scope of the systems and methods described herein, nor their applications, but merely provide an illustrative example of one embodiment.

The performance of the Probabilistic Search (PS) method is compared with Pan & Thompkins (PT) and other methods. The methods can be compared on ECG datasets with a total length of >80 hours. ECG with textile sensors in 3 daily states of the subjects are recorded: sitting, standing and jogging. Based on these original experimental recordings, a synthetic textile-ECG dataset was generated that fully characterizes the textile-ECG noise features. The original experimental recordings are a standardized textile-ECG dataset available for R-peak detection method validations. With the original experimental recordings, a Textile-like MITDB consisting of 108 30-minute recordings can be generated with varying SNR and levels of motion artifacts by adding modelled textile-ECG noise to 12 selected MITDB recordings. Thus, the synthetic dataset (Textile-like MITDB) can be standardized in the sense that the R-peak annotations are identical to the original MITDB, and these annotations can be fully justified by physicians. The R-peak detection accuracy of the methods in 3 datasets can be compared: (1) MITDB; (2) original experimentally recorded textile-ECG signals and (3) synthetic Textile-like MITDB.

In the following results on method comparisons, the terms true positive (TP), false positive (FP) and false negative (FN) are used to define accuracy measure statistics. The grace period for a detection can be the largest distance allowed between the detected R-peak and the corresponding annotated R-peak to be considered a TP. To be more specific, the time point (x) of the detected R-peak can be considered a TP if

$$\\ \begin{matrix}x ∈ [ y - Δ T , y + Δ T ] & ( 28 )\end{matrix}$$

where y can be the correct corresponding R-peak time in the annotation file, then ΔT can be called the grace period. The grace period (ΔT) can be specified for computing the R-peak accuracy measure statistics in each ECG dataset.
Exemplary Experimental Results with MITDB

MITDB is the standardized dataset to validate R-peak detection methods for clinical-ECG. The example PS method was tested on the full MITDB, which consists of the clinical-ECG recordings from 48 arrhythmia patients, and the length of each recording is 30.09 minutes. In the whole MITDB, there are in total˜105 annotated R-peaks, and the annotation was conducted by experienced physicians. As a commonly used standard accuracy measure statistic for MITDB, the total R-peak detection accuracy can be defined as

$$\\ 1 - \frac{FP + FN}{total annotation peaks} .$$

For MITDB, the commonly used grace period (defined by (28)) is 100≤ΔT≤150 ms. Here ΔT=100 ms was used to be consistent in comparisons. For the example PS method, the total R-peak detection accuracy was 99.85%, and this was the top performance among other methods on MITDB.

TABLE 1
MITDB benchmark in R-peak detections,
according to some embodiments.

| | Total true beats | | | Total |
|---|---|---|---|---|
| Method | (TP + FN) | FP | FN | accuracy (%) |
| Example PS (this work) | 109494 | 80 | 85 | 99.85 |
| ISEE | 109532 | 116 | 58 | 99.84 |
| WTSEE | 109494 | 99 | 79 | 99.84 |
| PSEE | 109494 | 91 | 93 | 99.83 |
| SEHT | 109496 | 140 | 79 | 99.80 |
| DOM | 109809 | 166 | 58 | 99.80 |
| STSE | 108494 | 97 | 171 | 99.75 |
| UNET | 109475 | 182 | 171 | 99.68 |
| Neural network | 105078 | 99 | 241 | 99.68 |
| WT | 109428 | 153 | 220 | 99.66 |
| PT | 109809 | 277 | 507 | 99.29 |

Exemplary Experimental Results with Synthetic Textile-Like MITDB

In order to validate R-peak detection methods on textile-ECG, a standardized Textile-like MITDB synthetic dataset was generated that fully characterizes the textile-ECG noise features from original experimental recordings. Textile-like MITDB consists of 108 signals, and each signal is of the same length as an original MITDB recording (30.09 minutes). 108 Textile-like MITDB signals are evenly classified into 9 categories based on the 3 types of noise and 3 levels of SNR (high, middle and low). The 3 noise types are high frequency (HF), mixed frequency (MF) and low frequency (LF). LF noise represents the motion artifacts, HF noise is mainly from the electrical circuits (e.g., power line interference), and MF noise has both LF and HF components. The recordings in each noise type are further classified into 3 SNR levels: high, middle (mid), and low. For the total recordings across all 3 SNR levels of a specific noise type, a one-way analysis-of-variance (ANOVA) test was implemented to compare the example PS method with the best performance of the other methods. The plots titled “All noise types” show comparison results in the whole Textile-like MITDB consisting of 108 30-minute recordings. Thus, Textile-like MITDB can incorporate the complete textile-ECG noise features, and this standardized dataset can be implemented to fully validate the R-peak detection methods.

An auto-regression model (AR(3)) was used to fit the noise and obtain the prediction residue and AR coefficients. The residue distribution was estimated by kernel density estimation (KDE), and a new residue sample was generated from KDE. The new residue sample was filtered with the same AR coefficients, and the new noise is generated.

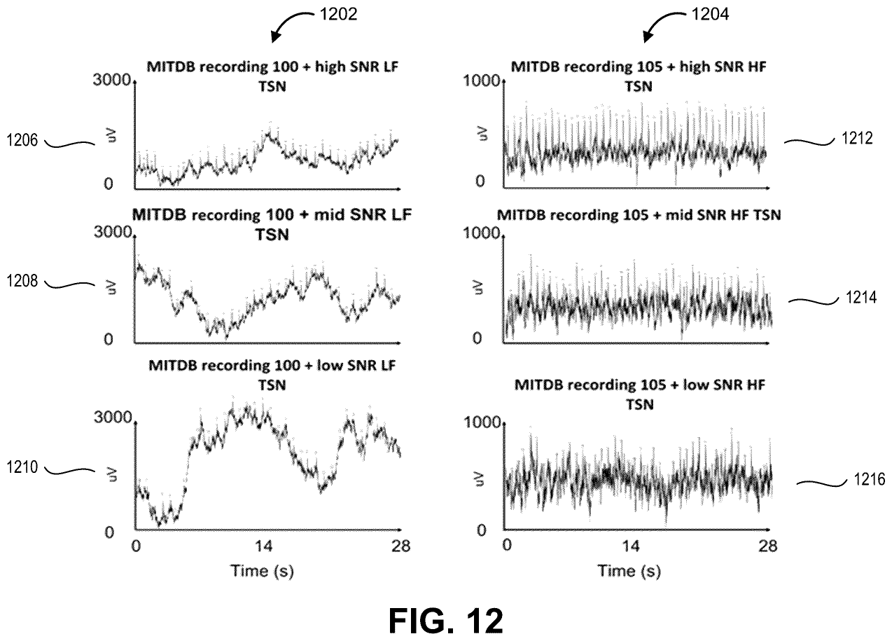

FIG. 12 illustrates the textile-like MITDB recordings, according to some embodiments.

Column 1202 illustrates additions of generated LF (low frequency) TSN's (textile sensor noise) to MITDB recording #100 (including high SNR (1206), middle SNR (1208), and low SNR (1210)). Column 1204 illustrates additions of generated HF (high frequency) TSN's to MITDB recording #105 (including high SNR (1212), middle SNR (1214), and low SNR (1216)). Note that both columns 1202 and 1204 have different scales in the y-axis. All the R-peaks are annotated with red circles on the top.

On Textile-like MITDB, the R-peak detection accuracy of the example PS method was compared with PT and 4 other methods, including Improved PT, Hamilton, Engelse, and Khamis. The following accuracy measure statistics can be used in the comparisons:
a) precision (or positive predictive value):

$$\\ \begin{matrix}PPV ( \% ) = \frac{TP}{TP + FP} * 100 & ( 29 )\end{matrix}$$

b) sensitivity:

$$\\ \begin{matrix}SEN ( \% ) = \frac{TP}{TP + FN} * 100 & ( 30 )\end{matrix}$$

c F1-score:

$$\\ \begin{matrix}F 1 ( \% ) = \frac{2 * TP}{( 2 * TP ) + FP + FN} * 100 & ( 31 )\end{matrix}$$

d) absolute heart rate estimate difference (AD) to the true heart rate (HR):

$$\\ \begin{matrix}AD ( bpm ) = \vert estimate HR - true HR \vert = \vert \frac{TP + FP}{total time {(}_{} = 30.09 minutes )} - true HR \vert & ( 32 )\end{matrix}$$

Heart rate estimate can be the output of many mobile applications. “bpm” can represent “beats per minute”. For each Textile-like MITDB signal, the true heart rate was computed using the R-peak annotation file of the corresponding original MITDB recording. F1-score can be the main statistic in the accuracy measures. The F1-score can represent harmonic mean of precision (PPV) and sensitivity (SEN) and can be used in validating signal detection methods. In computing TP, FP and FN, the grace period ΔT=50 ms (defined by (28)) can be used. The reason of using this stricter grace period (instead of ΔT=100 ms for MITDB) lies in the high noise level for textile-ECG. The smallest RR-interval clinically observed can be as low as 100 ms, so it may be reasonable to allow RR<200 ms for R-peak detection methods. The length of the tolerance interval can be 200 ms with ΔT=100 ms (Equation (28)). Since the noise level can be high in textile-ECG, the methods may choose two peaks (one R-peak, one noise) within a 200 ms tolerance interval. Thus, the additional noise peak can be considered as TP, and this may wrongly increase the accuracy statistics.

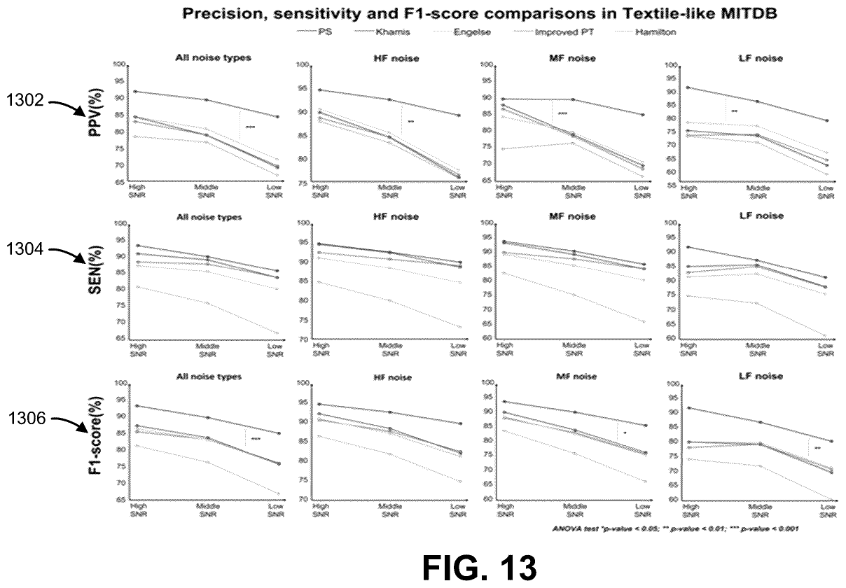

FIG. 13 illustrates comparisons of precision (PPV) (1302), sensitivity (SEN) (1304), and F1-score (1306) of the other ECG R-peak detection methods with the example Probabilistic Search (PS) method in the standardized Textile-like MITDB, according to some embodiments.

FIG. 13 shows that the example PS method was better than the best performance of the other 4 methods in each noise type for all 3 statistics (PPV, SEN and F1-score). The example PS method was better than the other methods in PPV and F1-score considering the whole Textile-like MITDB (“All noise types”, ANOVA, p<0.001).

The example PS method was better in PPV than the other methods for each noise type (ANOVA, p<0.01). The example PS method may be superior in PPV because of fewer FPs (Equation (29)), compared with the other methods. The reason that the example PS method induces fewer FPs may lie in its probabilistic and search mechanism. As shown in FIG. 3, in the R-peak detection step of the example PS method, the optimal time point around which the next R-peak is more probable can be predicted. Then the next R-peak can be searched for locally in detailed searching intervals with an increasing length, starting close to the optimal time point (see, e.g., FIG. 10). This searching method can reduce the amount of FPs, because it can avoid the less probable places where large noise peaks prevalently exist and mimic R-peaks.

TABLE 2
Precision (%) values of R-peak detection
methods, according to some embodiments.

| | HF | | | MF | | | LF | | |
|---|---|---|---|---|---|---|---|---|---|
| Methods | High | Mid | Low | High | Mid | Low | High | Mid | Low |
| PT | 71.8 | 59.77 | 49.3 | 87.25 | 55.15 | 46.2 | 57.61 | 52.81 | 44.55 |
| Improved | 88.75 | 84.73 | 76.76 | 86.56 | 78.32 | 68.36 | 73.63 | 73.98 | 64.43 |
| PT | | | | | | | | | |
| Hamilton | 88 | 83.54 | 76.04 | 74.46 | 76.22 | 66.09 | 73.31 | 71.03 | 59.13 |
| Engelse | 90.7 | 85.71 | 77.79 | 84.24 | 79.64 | 70.52 | 78.5 | 77.25 | 67.22 |
| Khamis | 89.9 | 84.68 | 76.19 | 87.79 | 78.8 | 69.34 | 75.38 | 73.47 | 62.42 |
| Example PS | 94.66 | 92.64 | 89.31 | 89.64 | 89.47 | 84.84 | 91.57 | 86.31 | 79.16 |

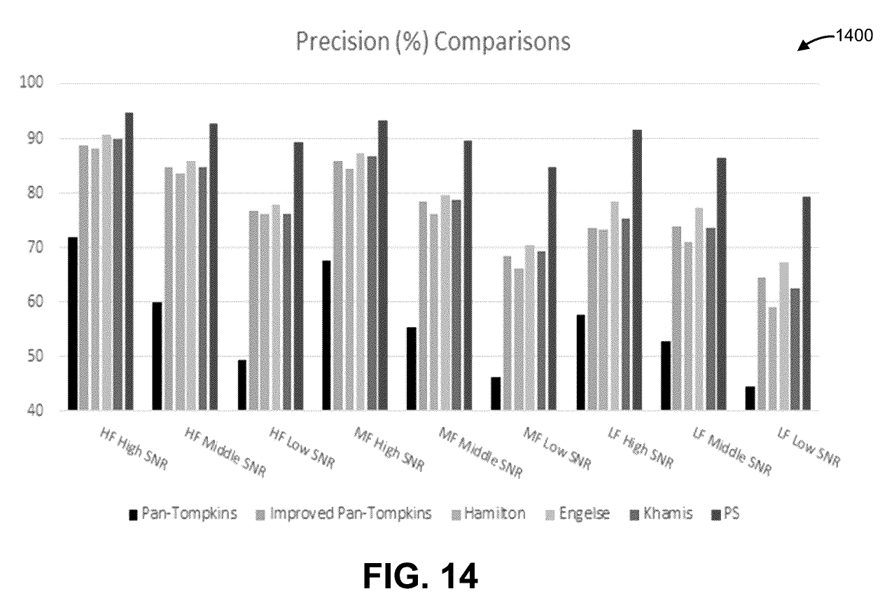

FIG. 14 illustrates a comparison of the precision (%) values of R-peak detection methods (1400), according to some embodiments.

The example PS method was better in SEN than the other methods for each noise type, although the comparisons may not be statistically significant. High SEN means that the number of missed R-peaks (FN) is small. This shows that the example PS method can be effective in catching all R-peaks. The other methods are significantly worse than the example PS method in PPV. The low PPV of the other methods demonstrates that they may create large amounts of FPs during the R-peak detection. Thus, the other methods can often follow a conservative searching strategy, trying best to catch all R-peaks by searching in an interval as large as possible. This strategy may be helpful for R-peak detection in high quality clinical-ECG with a high SNR, but may not be appropriate for textile-ECG with prevalent and large noise.

TABLE 3
Sensitivity (%) values of R-peak detection
methods, according to some embodiments.

| | HF | | | MF | | | LF | | |
|---|---|---|---|---|---|---|---|---|---|
| Methods | High | Mid | Low | High | Mid | Low | High | Mid | Low |
| PT | 92.29 | 89.98 | 87.54 | 91.42 | 88.38 | 84.53 | 84.71 | 86.05 | 80.32 |
| Improved | 92.45 | 90.75 | 89.04 | 89.77 | 87.53 | 84.04 | 82.84 | 84.89 | 77.72 |
| PT | | | | | | | | | |
| Hamilton | 84.88 | 80.15 | 73.37 | 82.66 | 75.11 | 65.86 | 74.77 | 72.28 | 61.07 |
| Engelse | 91.08 | 88.52 | 84.71 | 89.13 | 85.36 | 80.22 | 81.36 | 82.36 | 75.41 |
| Khamis | 94.55 | 92.38 | 88.65 | 93.06 | 89.07 | 84.02 | 84.99 | 85.52 | 77.91 |
| Example PS | 94.7 | 92.55 | 89.95 | 93.6 | 90.22 | 85.68 | 91.72 | 87.16 | 81.16 |

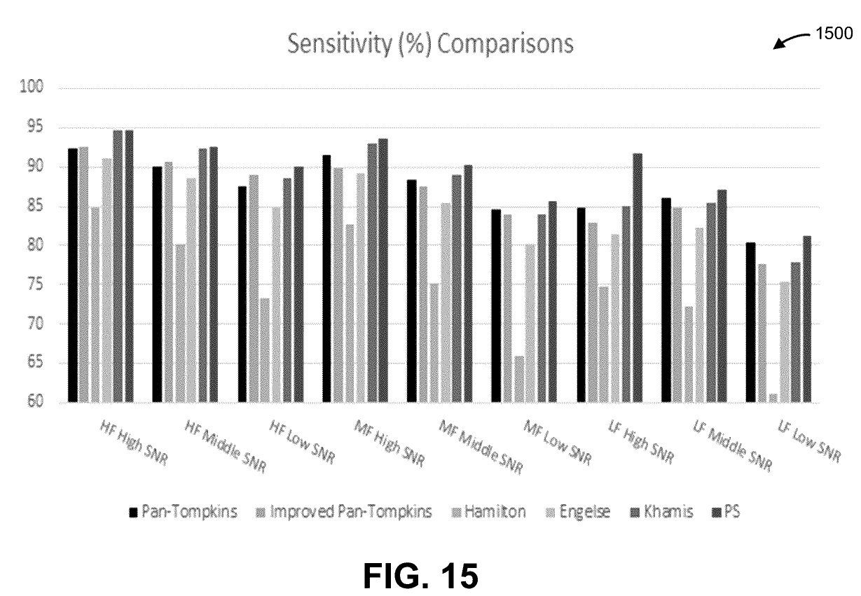

FIG. 15 illustrates a comparison of the sensitivity (%) values of R-peak detection methods (1500), according to some embodiments.

F1-score is the harmonic mean of PPV and SEN and can be the main statistic that represents the overall accuracy of a signal detection method. The example PS method was >20% higher than PT in F1-score for each noise type.

TABLE 4
F1-score (%) values of R-peak detection
methods, according to some embodiments.

| | HF | | | MF | | | LF | | |
|---|---|---|---|---|---|---|---|---|---|
| Methods | High | Mid | Low | High | Mid | Low | High | Mid | Low |
| PT | 80.76 | 71.83 | 63.08 | 77.79 | 67.91 | 59.75 | 68.58 | 65.45 | 57.32 |
| Improved | 90.56 | 87.64 | 82.44 | 87.82 | 82.67 | 75.39 | 77.97 | 79.06 | 70.45 |
| PT | | | | | | | | | |
| Hamilton | 86.41 | 81.81 | 74.68 | 83.48 | 75.66 | 65.98 | 74.03 | 71.65 | 60.08 |
| Engelse | 90.89 | 87.09 | 81.1 | 88.2 | 82.34 | 75.06 | 79.91 | 79.72 | 71.08 |
| Khamis | 92.17 | 88.36 | 81.95 | 89.8 | 83.62 | 75.98 | 79.89 | 79.04 | 69.31 |
| Example PS | 94.67 | 92.59 | 89.63 | 93.48 | 89.83 | 85.25 | 91.64 | 86.72 | 80.13 |

TABLE 5
Average F1-score (%) values of each noise
type, according to some embodiments.

| | Average HF | Average MF | Average LF |
|---|---|---|---|
| Methods | noise | noise | noise |
| PT | 71.89 | 68.48 | 63.78 |
| Improved PT | 86.88 | 81.96 | 75.83 |
| Hamilton | 80.97 | 75.04 | 68.59 |
| Engelse | 86.36 | 81.87 | 76.90 |
| Khamis | 87.49 | 83.13 | 76.08 |
| Example PS | 92.30 | 89.52 | 86.16 |
| Difference between | 4.81 | 6.39 | 10.08 |
| Example PS and | | | |
| Khamis | | | |

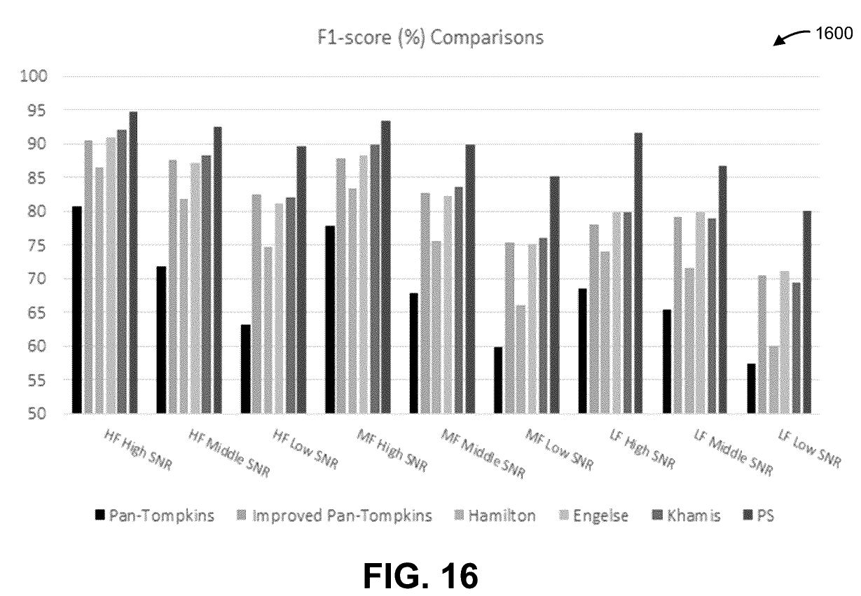

FIG. 16 illustrates F1-score (%) values of R-peak detection methods (1600) (Table 4), according to some embodiments.

In FIG. 13, performance of the example PS method is shown being possibly better in F1-score than the other methods for each noise type and the comparison may be statistically significant in MF noise (ANOVA, p=0.013<0.05) and LF noise (ANOVA, p=0.006<0.01). In HF noise, the example PS method was better than this experiment's best of the other methods (Khamis), though the difference may not reach significance (ANOVA, p=0.068). Motion artifact (LF noise) was the most challenging noise type for the ECG R-peak detection, because it can be difficult to efficiently remove with the current band-pass filters. This is shown wherein as the level of motion artifact increases, p-value decreases, possibly meaning that the advantage of the example PS method over the other methods becomes more significant. The F1-score of the example PS was ˜10% better than this experiment's best of the other methods (Khamis) in signals with LF noise. This shows that the example PS R-peak detection method can be highly robust to motion artifacts.

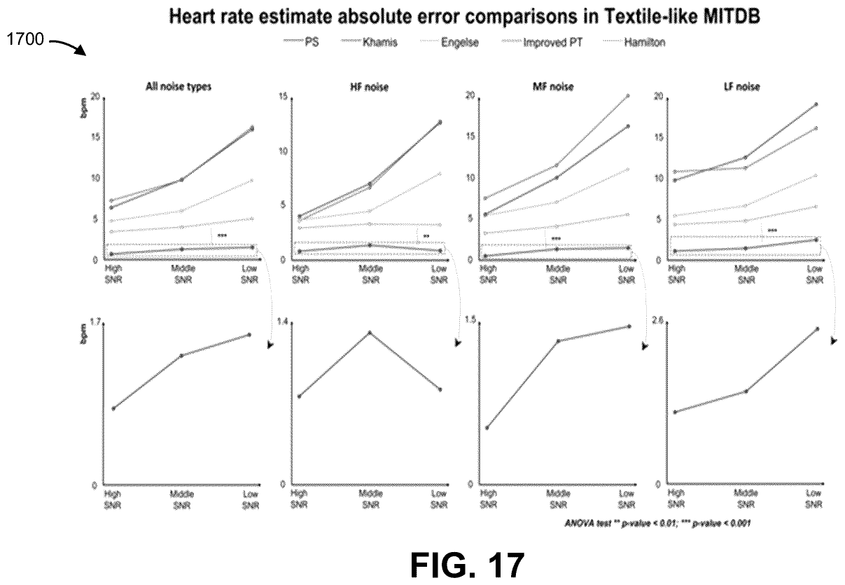

FIG. 17 illustrates comparisons of heart rate estimate absolute error of the other ECG R-peak detection methods with the example Probabilistic Search (PS) method in the standardized Textile-like MITDB (1700), according to some embodiments.

FIG. 17 shows that for the heart rate estimate, the example PS method was better than the best performance of the other 4 methods in the whole Textile-like MITDB (“All noise types”, ANOVA, p<0.001), as well as for each noise type (“HF”, p<0.01; “MF”, p<0.001; “LF”, p<0.001). Though the Hamilton method also performs well in estimating heart rate, its R-peak detection accuracy (F1-score) was below the other methods (FIG. 13). In the Textile-like MITDB (54 hours of recordings), the average heart rate estimate absolute error with the example PS method was only 1.29 bpm. Thus, the example PS R-peak detection method may be an optimal choice for mobile applications in daily heart rate monitoring with textile-ECG in the experiments carried out.

TABLE 6
Heart rate estimate absolute error (bpm), according to some embodiments.

| | HF | | | MF | | | LF | | | Total |
|---|---|---|---|---|---|---|---|---|---|---|
| Methods | High | Mid | Low | High | Mid | Low | High | Mid | Low | Average |
| PT | 21.93 | 38.84 | 59.6 | 26.93 | 46.32 | 63.75 | 36.14 | 48.38 | 61.69 | 44.84 |
| Improved | 3.6 | 6.63 | 12.7 | 7.52 | 11.53 | 19.96 | 10.87 | 11.32 | 16.17 | 11.14 |
| PT | | | | | | | | | | |
| Hamilton | 2.93 | 3.3 | 3.22 | 3.31 | 4.15 | 5.58 | 4.43 | 4.87 | 6.61 | 4.27 |
| Engelse | 3.65 | 4.46 | 7.91 | 5.44 | 7.05 | 11.04 | 5.51 | 6.72 | 10.43 | 6.91 |
| Khamis | 4 | 6.99 | 12.57 | 5.58 | 10.02 | 16.26 | 9.8 | 12.6 | 19.07 | 10.77 |
| Example PS | 0.78 | 1.33 | 0.84 | 0.54 | 1.36 | 1.5 | 1.18 | 1.52 | 2.55 | 1.29 |

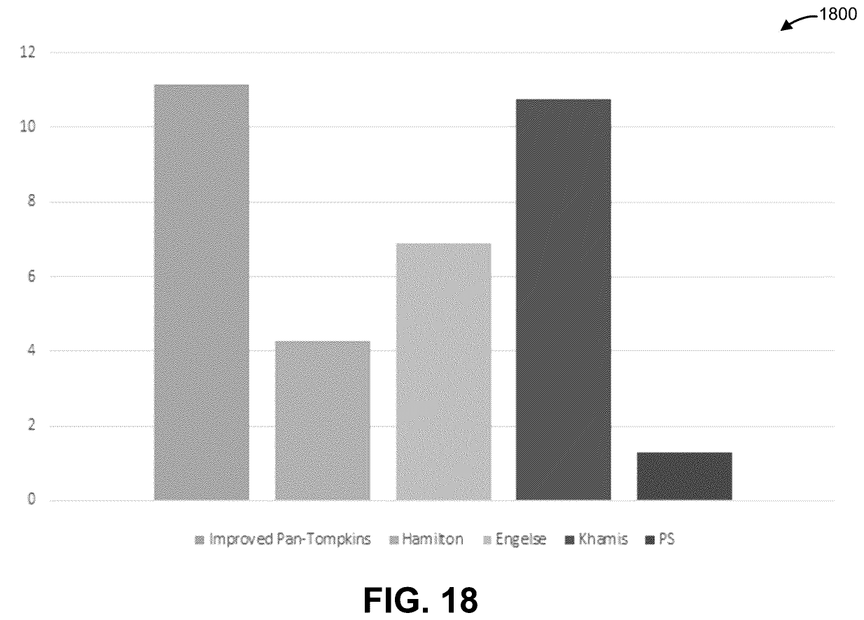

FIG. 18 illustrates the average heart rate estimate absolute error (bpm) in the whole Textile-like MITDB (PT excluded) (1800) (Table 6), according to some embodiments.

Exemplary Experimental Results with Original Textile-ECG Dataset

Besides the analysis on the standardized Textile-like MITDB synthetic ECG dataset, the R-peak detection methods were compared in an original textile-ECG dataset. ECG data was recorded with textile sensors on the waist in 3 daily states: sitting, standing and jogging. From other textile-ECG datasets, the signals from the 7 subjects recorded with active electrode were selected. Waist-recording can test the potential of textile ECG sensors to be implemented in smart underwear for daily health monitoring. The ECG signals recorded with active electrodes were selected from textile-ECG datasets. The active electrode has built-in readout circuitry, and can be implemented in daily wearable healthcare due to its robustness to environmental noise.

The 7 subjects were all healthy, with 4 males, 3 females, with an average age of 25.7 years. For each subject, 3 moving states (sitting, standing and jogging) were recorded, 2 electrode materials (carbon and silver) and 4 locations on the waist. Each textile recording condition is a combination of moving states, electrode materials and waist-recording locations. The total number of such combinations was 3*2*4=24. For each of the 7 subjects, one trial (˜100 seconds) for each of the 24 textile recording conditions was recorded, thus the total number of trials was 7*24=168.

A one-way analysis-of-variance (ANOVA) test was implemented to validate the statistical significance of the F1-score comparisons of the example PS method with the other methods.

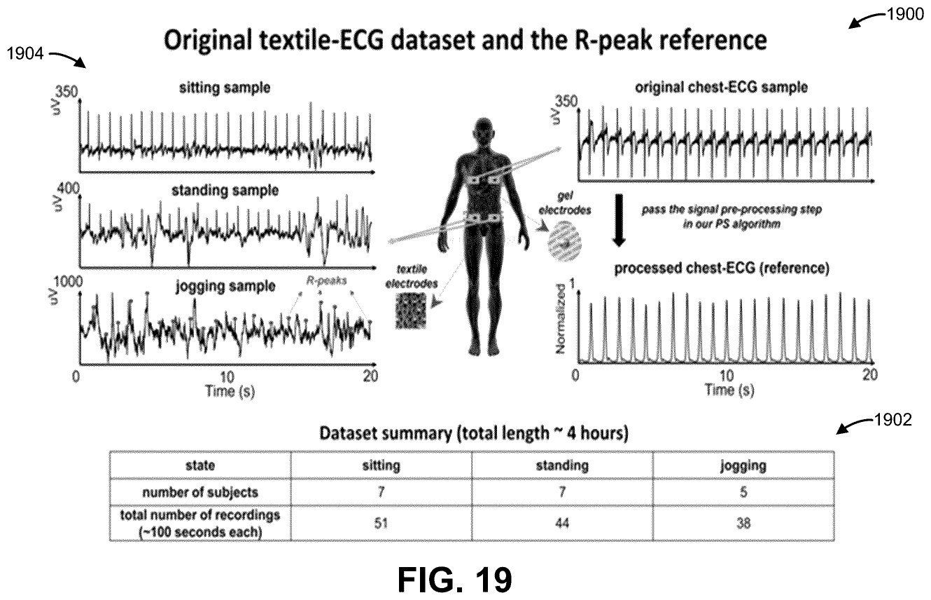

FIG. 19 illustrates the textile-ECG dataset and the reference chest R-peaks, according to some embodiments.

During each textile-ECG recording, a reference ECG signal with gel-electrodes from the chest was simultaneously recorded. This reference chest-ECG signal (see FIG. 19 for an example 1900) has high quality (large SNR), and has the same R-peaks (i.e., heart beats) as the simultaneously recorded textile-ECG (1904). Then, the SNR of this chest-ECG was further amplified with the signal pre-processing method (see, e.g., FIG. 3) used for the example PS method. Finally, the R-peaks in the pre-processed chest-ECG were identified with a single threshold, and these R-peaks are used as the reference (ground truth) to compute the accuracy measure statistics. The R-peaks in the chest-ECG can be perfectly detected with the example PS method, and can be used as the reference to compute the accuracy measure statistics. The jogging sample is contaminated with motion artifacts, and the R-peaks in this noisy textile-ECG are annotated with the corresponding chest-ECG reference signal. The whole original textile-ECG dataset (˜4 hours long) is summarized in the bottom table 1902 of FIG. 19.

From the total 168 textile-ECG trials, the trials without the perfect chest-ECG reference were excluded. For the jogging state, the recordings from 2 subjects were excluded because they all have bad references due to the inappropriate contacts of the gel-electrodes with the chest skin. The remaining 133 trials constitute the textile-ECG dataset used in this work and were used for the data analysis.

FIG. 19 shows that the textile-ECG in steady states (sitting and standing) may have fewer motion artifacts (e.g., from moving hands and legs, switching body weight lightly from left to right), but the jogging state signal (performed on a treadmill at ˜2 mph) is contaminated with high motion artifacts that mimic or even surpass the R-peaks. Thus, the motion artifacts in the jogging textile-ECG may increase the difficulty of R-peak detection. In the original textile-ECG dataset, the R-peak detection F1-score of 3 methods was compared: PT, the example PS method and Khamis. F1-score represents the overall accuracy of a signal detection method well. Khamis was selected because it performed better than the other methods in terms of F1-score in the standardized Textile-like MITDB (see FIG. 13). As in Textile-like MITDB, a grace period ΔT=50 ms (defined by (28)) was used in computing TP, FP and FN for the F1-score (defined by (31)).

TABLE 7
F1-score (%) comparisons in the original textile-
ECG dataset, according to some embodiments.

| | State | | | | | |
|---|---|---|---|---|---|---|
| | Sitting | | Standing | | Jogging | |
| | Statistics | | | | | |
| | | Standard | | Standard | | Standard |
| Method | Mean | Deviation | Mean | Deviation | Mean | Deviation |
| PT | 86.37 | 27.39 | 79.83 | 15.28 | 79.21 | 20.44 |
| Khamis | 95.13 | 7.03 | 92.72 | 10.64 | 79.57 | 16.4 |
| Example PS | 99.34 | 0.99 | 99.05 | 1.79 | 92.62 | 8.25 |

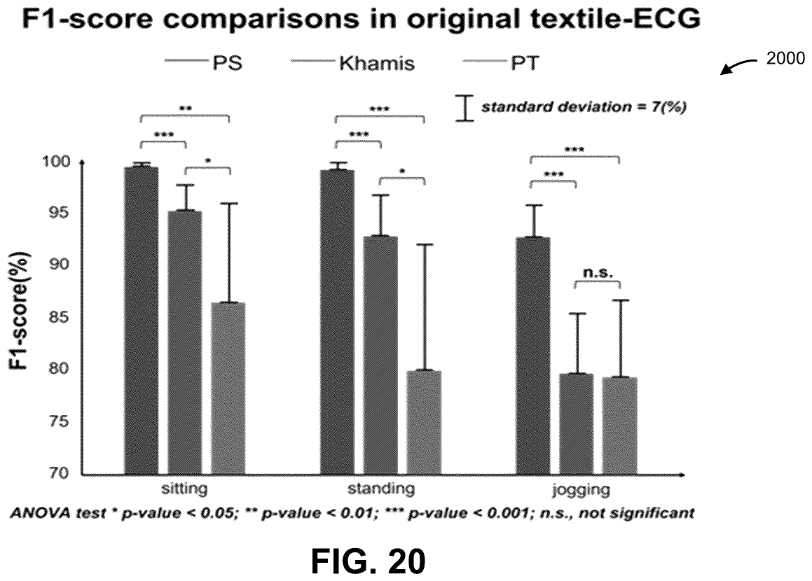

FIG. 20 illustrates the comparisons of both mean and standard deviation of the F1-score (2000), according to some embodiments. The height of the legend for the standard deviation represents that the F1-score sample standard deviation=7(%).

FIG. 20 shows that the example PS method was better than the other methods in all 3 daily textile-ECG recording states: sitting, standing, and jogging (ANOVA, p<0.01, all comparisons). FIG. 20 also shows that though the Khamis method was better than PT in sitting and standing (ANOVA, 0.01<p<0.05, both comparisons), its performance was close to PT (ANOVA, not significant) in the jogging state. Compared with PT, Khamis may fail in improving the R-peak detection accuracy of the original textile-ECG signals recorded in the jogging state. These signals may be contaminated with motion artifacts and have a very low SNR. In these jogging state signals, the F1-score of the example PS method was 92.6%, whereas both Khamis and PT was <80% (see Table 7). In all comparisons, the standard deviation of the example PS method was smaller than the other methods. This shows that the example PS method can be highly robust to all types of textile sensor noise, including both high frequency noise (exists in all 3 states) and motion artifacts (prevalent in the jogging signals). Thus, the example PS method may be the optimal choice for the R-peak detection in textile-ECG considering both accuracy and noise robustness.

Conclusions from the Experimental Results

The Probabilistic Search (PS) can accurately and robustly detect R-peaks from noisy ECG signals recorded by textile sensors. Motion artifacts are a typical feature of textile-ECG, as well as other wearable-ECG conveniently recorded for the purpose of daily heart rate monitoring. Compared with the PT and 4 other R-peak detection methods in textile-ECG datasets, the example PS method was superior in R-peak detection accuracy (F1-score) and had higher robustness to the textile motion artifacts. In a 54-hour standardized textile-ECG dataset that fully characterizes textile noise features, the average heart rate estimate error was only 1.29 bpm with the example PS method. This means that a PS method may be an advantageous solution in convenient heart rate monitoring with wearable-ECG recorded by, for example, mobile applications. Besides heart rate estimation, R-peak detection may also be the first step of further classifying ECG signals and obtaining valuable medical information. The PS R-peak detection method may be an advantageous choice for daily heart health monitoring and analysis with ECG recorded by wearable sensors.

### Other Applications

Many further features and combinations thereof concerning embodiments described herein may be apparent to those skilled in the art following a reading of the instant disclosure.

For example, the membrane potential of the heart's pacemaker cell can be depolarized by ion currents (e.g., Ca²⁺, K⁺, and Na⁺) similar to the integrate-and-fire activity of neurons. Such integrate-and-fire process of the membrane potential dynamics can be modelled as a random walk stochastic process like that with a firing threshold and a drifting term to an equilibrium. The first passage time (FPT) means the time difference between two adjacent threshold-crossing events. The FPT probability density of such an integrate-and-fire drifting random walk process is derived as Inverse Gaussian. Inverse Gaussian distributions can be used to study both firing neurons and heart beats.

### Implementation Details

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references were made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The foregoing discussion provides many example embodiments. Although each embodiment represents a single combination of inventive elements, other examples may include all possible combinations of the disclosed elements. Thus, if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

For simplicity only one computing device 604 was shown in FIG. 6, but system 600 may include more computing devices 604 operable by users to access remote network resources and exchange data. The computing devices 604 may be the same or different types of devices. The computing device 604 at least one processor, a data storage device (including volatile memory or non-volatile memory or other data storage elements or a combination thereof), and at least one communication interface. The computing device components may be connected in various ways including directly coupled, indirectly coupled via a network, and distributed over a wide geographic area and connected via a network (which may be referred to as “cloud computing”).

For example, and without limitation, the computing device may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein

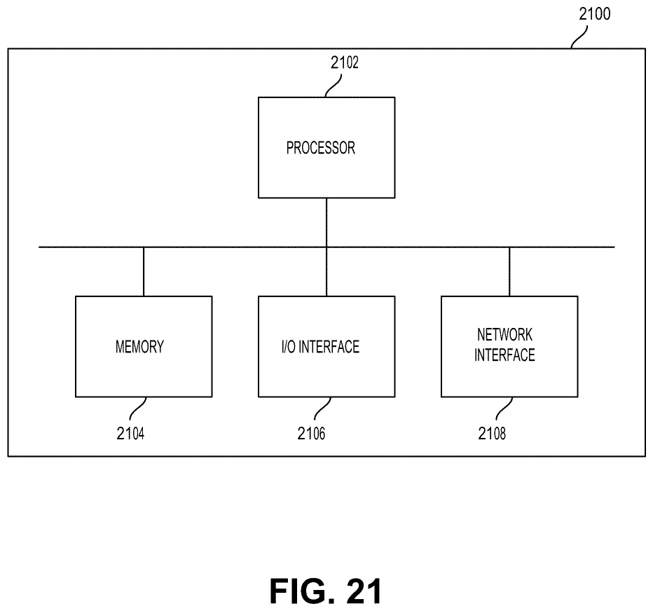

FIG. 21 is a schematic diagram of computing device 2100, exemplary of an embodiment. Computing device 2100 can be used for computing device 604 in FIG. 6. As depicted, computing device 2100 includes at least one processor 2102, memory 2104, at least one I/O interface 2106, and at least one network interface 2108.

Each processor 2102 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 2104 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 2106 enables computing device 2100 to interconnect with one or more input devices, such as a keyboard, mouse, camera, touch screen and a microphone, or with one or more output devices such as a display screen and a speaker.

Each network interface 2108 enables computing device 2100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 2100 is operable to register and authenticate users (using a login, unique identifier, and password for example) prior to providing access to applications, a local network, network resources, other networks and network security devices. Computing devices 2100 may serve one user or multiple users.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only. The scope is indicated by the appended claims.

What is claimed is:

1. A system for probabilistic search for R-peak detection from electrocardiogram (ECG), the system comprising:
a communication interface to receive a data stream comprising an ECG signal;
a processing system with one or more hardware processors and one or more memories storing executable instructions for R-peak detection and heart rate monitoring, wherein the hardware processor programmed with the executable instructions:
pre-processes and smooths the data stream comprising the ECG signal to reduce noise in the data stream and amplify the ECG signal;
detects R-peaks in an initial time interval of the pre-processed data stream;
generates a predicted time point of a first new R-peak by predicting a probabilistic distribution of the new R-peak following a last R-peak in the initial time interval with History Dependent Inverse Gaussian (HDIG) distribution, wherein distribution parameters are updated in real-time based on recent R-peak history;
detects the first new R-peak in the data stream by searching for the first new R-peak around the predicted time point of the first new R-peak in an adaptive searching interval, wherein the peak with a largest amplitude passing a varying adaptive threshold is detected as the first new R-peak,
detects remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, wherein peaks with largest amplitudes passing the varying adaptive threshold are detected as the remaining R-peaks; and
compute heart rate metrics using the detected R-peaks, the heart rate metrics being one or more of average heart rate, accuracy, F1-score, sensitivity, precision, and heart rate variability;
non-transitory memory for storing the executable instructions for the R-peak detection; and
an output device for transmitting and/or storing the heart rate metrics.

2. The system of claim 1 wherein the hardware processor measures the average heart rate in the data stream as a total number of detected R-peaks in the data stream divided by a time length of the data stream.

3. The system of claim 1 wherein the hardware processor is programmed with executable instructions for continuous heart rate monitoring by continuously receiving the data stream and re-computing the heart rate metrics.

4. The system of claim 1, further comprising a band-pass filter to pre-process the data stream to reduce baseline wander and high frequency noise.

5. The system of claim 1, wherein the hardware processor pre-processes and smooths the data stream using an energy calculator that amplifies the R-peaks in the ECG signal.

6. The system of claim 1, wherein the hardware processor pre-processes and smooths the data stream using a smoothing function that convolves the data stream with a Gaussian kernel specific to a sampling frequency of the data stream and a type of sensor recording the data stream to further increase the signal-to-noise ratio (SNR).

7. The system of claim 1, wherein the hardware processor pre-processes and smooths the data stream with a Gaussian kernel by assigning higher weights to R-peaks in the ECG signal and lower weights to noise in the data stream.

8. The system of claim 1, wherein distribution parameters are updated in real-time based on recent R-peak history with a fast optimization technique such that more recent R-peak history has higher weights than more previous R-peak history.

9. The system of claim 1, wherein the adaptive searching interval depends on the predicted time point of the first new R-peak.

10. The system of claim 1, wherein the adaptive searching interval depends on patient specific skew parameters based on an extent of arrhythmia selected from the group consisting of slight arrhythmia, moderate arrhythmia, and serious arrhythmia.

11. The system of claim 1, wherein the adaptive searching interval is at an interval of increasing length.

12. The system of claim 1, wherein the varying adaptive threshold is specific to a type of sensor recording the data stream.

13. The system of claim 1, wherein the adaptive searching interval is generated by training on datasets based on a large amount of ECG data.

14. The system of claim 1, comprising one or more electrodes to perform measurements to record a raw ECG signal for the data stream comprising the ECG signal.

15. The system of claim 14, wherein the electrodes are selected from the group of dry-electrodes and gel-electrodes.

16. The system of claim 1, comprising one or more wearable sensors to perform measurements to record the raw ECG signal.

17. The system of claim 16, wherein the one or more wearable sensors comprise one or more wearable textile sensors.

18. The system of claim 1, wherein the HDIG distribution extracts an underlying probabilistic structure of R-peak positions in the data stream based on deterministic morphological features of ECG signals.

19. The system of claim 1, wherein model parameters for the HDIG distribution are updated in real-time.

20. A method for probabilistic search for R-peak detection from electrocardiogram (ECG), the method comprising:
pre-processing and smoothing a data stream comprising an ECG signal to reduce noise in the data stream and amplify the ECG signal;
detecting R-peaks in an initial time interval of the pre-processed data stream;
generating a predicted time point of a first new R-peak by predicting a probabilistic distribution of the new R-peak following a last R-peak in the initial time interval with History Dependent Inverse Gaussian (HDIG) distribution, wherein distribution parameters are updated in real-time based on recent R-peak history;
detecting the first new R-peak in the data stream by searching for the first new R-peak around the predicted time point of the first new R-peak in an adaptive searching interval, wherein the peak with a largest amplitude passing a varying adaptive threshold is detected as the first new R-peak;
detecting remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, wherein peaks with largest amplitudes passing the varying adaptive threshold are detected as the remaining R-peaks; and
computing heart rate metrics using the detected R-peaks, the heart rate metrics being one or more of average heart rate, accuracy, F1-score, sensitivity, precision, and heart rate variability.

21. A non-transitory computer readable medium with instructions for R-peak detection from electrocardiogram (ECG) stored thereon, that when executed by a hardware processor cause the processor to:
pre-process and smooth a data stream comprising the ECG signal to reduce noise in the data stream and amplify the ECG signal;
detect R-peaks in an initial time interval of the pre-processed data stream;
generate a predicted time point of a first new R-peak by predicting a probabilistic distribution of the new R-peak following a last R-peak in the initial time interval with History Dependent Inverse Gaussian (HDIG) distribution, wherein distribution parameters are updated in real-time based on recent R-peak history;
detect the first new R-peak in the data stream by searching for the first new R-peak around the predicted time point of the first new R-peak in an adaptive searching interval, wherein the peak with a largest amplitude passing a varying adaptive threshold is detected as the first new R-peak;
detect remaining R-peaks following the first new R-peak in the data stream iteratively and in real-time as the data stream is received, by, for each of the remaining R-peaks, generating a predicted time point of an R-peak following a most recently detected R-peak by predicting an HDIG distribution of the R-peak following the most recently detected R-peak, and searching in the adaptive searching interval around the predicted time point, wherein peaks with largest amplitudes passing the varying adaptive threshold are detected as the remaining R-peaks; and
compute heart rate metrics using the detected R-peaks, the heart rate metrics being one or more of average heart rate, accuracy, F1-score, sensitivity, precision, and heart rate variability.
